(19) United States
(12) Patent Application Publication
(10) Pub. No.: US 2018/0161088 A1
(43) Pub. Date: Jun. 14, 2018

(54) ARTHROSCOPIC DEVICES AND METHODS

(71) Applicant: RELIGN Corporation, Cupertino, CA (US)

(72) Inventors: Ben Poser, Campbell, CA (US); Aaron Germain, San Jose, CA (US); Simon Malkevich, Gilroy, CA (US)

(73) Assignee: RELIGN Corporation, Cupertino, CA (US)

(21) Appl. No.: 15/659,241

(22) Filed: Jul. 25, 2017

Related U.S. Application Data
(60) Provisional application No. 62/366,512, filed on Jul. 25, 2016.

(51) Int. Cl. A61B 18/14

(52) U.S. Cl. CPC A61B 18/148; A61B 2018/00607

(57) ABSTRACT

An arthroscopy handpiece includes a handle body and a plurality of electrical components carried by the handle body. The electrical components may be connected to an external controller and power supply remote from the handle body. Typical electrical components include a motor and radiofrequency (RF) contacts adapted for coupling to a disposable electrosurgical tool. The handle body is usually both thermally and electrically conductive, and insulator elements are disposed between the electrical components and the handle body, with each insulator element adapted to prevent a potential leakage current flow from an electrical component to the handle body to prevent accidental electrical shock to the user.

### CROSS-REFERENCE TO RELATED APPLICATIONS

This application claims the benefit of provisional application no. 62/366,512 (Attorney Docket No. 41879-727.101), filed on Jul. 25, 2016, the full disclosure of which is incorporated herein by reference.

### BACKGROUND OF THE INVENTION

### 1. Field of the Invention

This invention relates to arthroscopic tissue cutting and removal devices by which anatomical tissues may be cut and removed from a joint or other site. More specifically, this invention relates to instruments configured for cutting and removing soft tissue with an electrosurgical device.

In several surgical procedures including subacromial decompression, anterior cruciate ligament reconstruction involving notchplasty, and arthroscopic resection of the acromioclavicular joint, there is a need for cutting and removal of bone and soft tissue. Currently, surgeons use arthroscopic shavers and burrs having rotational cutting surfaces to remove hard tissue in such procedures.

Many present arthroscopic and other surgical devices comprise handles configured to interchangeably receive different powered tools. Often the tools are motor-driven and optionally carry radiofrequency electrodes and other energized interfaces. As the handles will typically receive and control electrical energy from an external controller and power supply, significant waste heat can be generated in the handle and must be removed.

The need exists for arthroscopic cutters and other tools that can be interchangeably connected to handles and other handpieces, where the handpieces are fabricated from thermally conductive materials, such as thermally conductive metals, and in particular from aluminum. Such thermally conductive handles and handpieces must, however, be safe for manual use and in particular must the safely electrically insulated to inhibit current leakage and possible electrical shocks. At least some of these objectives will be met by the inventions herein.

### 2. Description of the Background Art

Related commonly owned US patents include: Pat. Nos. 9,585,675;; 9,603,656; and 9,681,913; and related commonly owned co-pending applications Ser. Nos. 14/990,610; 15/045,055; 15/271,184; 15/271,187; 15/410,723; 15/415,721; 15/818,495; 15/421,264; 15/449,796; 15/454,690; 15/483,940; 15/495,620; 15/599,372; and 15/633,372, the full disclosures of which are incorporated herein by reference.

### SUMMARY OF THE INVENTION

According to the present invention, an arthroscopy handpiece comprises a handle body and a plurality of electrical components carried by the handle body. An electrical cable typically connects each electrical component to an external controller and power supply disposed remotely from the handle body. The electrical components typically include at least a motor and one or more radiofrequency (RF) contacts that are adapted for coupling to a disposable electrosurgical tool. A plurality of insulator elements are disposed between the electrical components and the handle body, with at least one insulator element adapted to prevent or inhibit potential leakage of current flows from each of the electrical components to the handle body. Such potential leakage flows are preferably limited to threshold currents below 5 mA, 10 mA, 25 mA, 50 mA, 100 mA or 200 mA.

In some variations, the RF contacts may comprise first and second opposing polarity RF contacts adapted for coupling to a disposable bi-polar electrosurgical tool. In some variations, the motorized handpiece may have additional electrical components such as electrical switches, a control panel (including for example, an electronic display, electrical on/off switches, a joystick, or the like), one or more Hall sensors, and one or more Schmitt triggers.

In another variation, an insulator element can comprise an anodized layer formed over an electrically conductive material, typically an anodized metal layer formed over a surface of an electrically conductive metal, such as an anodized aluminum layer formed by anodizing a surface of an aluminum handle, an aluminum motor housing, or other structural component. The insulator element or feature also can comprise a polymeric layer, a ceramic layer, and/or an air gap disposed between the handle and electrical components, and the like.

In a first aspect of the present invention, a motorized handpiece includes a handle body having an exterior configured to be manually grasped and an interior. Electrical components including, for example, a motor and an RF contact are disposed within the interior of the handle body. The handle body is formed at least in part from a material which is both thermally and electrically conductive. In this way, the heat generated by the electrical components in the interior of the handle body can be rapidly dissipated through the handle body having relatively high thermal conductivity. As the handle body is both thermally and electrically conductive, it will be necessary to electrically isolate the electrical components within the housing to prevent or inhibit potential leakage currents from the electrical components from passing in the handle body. Insulator elements, as described in more detail below, will be disposed between each of the electrical components and adjacent surfaces of the interior of the handle body. In this way, any leakage current from a fault in an electrical component into the handle body is inhibited or prevented.

In specific embodiments of the motorized handpieces of the present invention, the insulator elements are adapted to inhibit a potential leakage current above a threshold value of 5 mA, usually 10 mA, frequently 25 mA, often at least 50 mA, sometimes at least 100 mA, and other times at least 200 mA, or even higher. In other specific embodiments, the motorized handpieces of the present invention will further comprise an electrical cable extending from the handle body. The cable will be typically be configured to connect each electrical component to the external controller and/or power supply.

In exemplary embodiments, the RF contacts may comprise first and second opposing polarity RF contacts, where the opposing RF contacts are typically adapted for coupling to bipolar contacts on a disposable electrosurgical tool when the disposable electrosurgical tool is coupled to the motorized handpiece. Other exemplary electrical components include one or more electrical switches, a control panel, one or more Hall sensors, one or more Schmitt triggers, and the like. The control panel may include a variety of components such as display panels, a joy stick, and electrical switches.

The insulator elements of the present invention may have a variety of forms. A preferred insulator element comprises a layer of an anodized material formed over at least a portion of the handle body or other component of the motorized handpiece. For example, the handle body will often be formed from a thermally conductive metal, such as aluminum where the electrical insulation can be provided by anodizing at least a portion of the surface of the aluminum or other metal handle body. Other suitable insulator elements include polymeric materials, ceramic materials, O-rings, and the like.

In further specific embodiments, a shell of the motor within the interior of the handle body may also be formed from aluminum. The aluminum shell may also have an anodized layer of aluminum formed there over. Anodized metals, while retaining most or all of their thermal conductivity, provide a highly effective electrically insulative layer which isolates the handle, motor, or other component from adjacent components within the handle body.

In a second aspect of the present invention, an arthroscopy handpiece comprises a handle body, motor, and electrical insulation between the handle body and the motor. The motor is typically carried in an interior of the handle body, and the handle comprises an electrically conductive material having a thermal conductivity of at least 50 W/(MK), usually at least 100 W/(MK), to carry heat away from the motor. The electrical insulation is configured to inhibit a potential leakage current from the motor to the electrically conductive handle body.

Other specific aspects of the arthroscopy handpiece will be the same as described previously for the motorized handpieces of the present invention.

In a third aspect of the present invention, a motorized arthroscopy handpiece comprises a handle body, an electronic control panel carried by the handle body, and at least one insulator disposed between the display and the handle body. The at least one insulator will be adapted or configured to prevent a leakage current from the display to the handle body above a pre-selected threshold value.

At least one insulator can be configured or selected to provide a maximum pre-selected threshold value of the leakage current of 5 mA, often 10 mA, usually 25 mA, in some cases 50 mA, in other cases 100 mA, and in still further cases 200 mA, or above. Other aspects of the motorized arthroscopy handpiece will be similar or identical to features described previously with respect to the motorized handpiece and the arthroscopy handpiece above.

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1 is a perspective view of a disposable arthroscopic cutter or burr assembly with a ceramic cutting member carried at the distal end of a rotatable inner sleeve with a window in the cutting member proximal to the cutting edges of the burr.

FIG. 2 is an enlarged perspective view of the ceramic cutting member of the arthroscopic cutter or burr assembly of FIG. 1.

FIG. 3 is a perspective view of a handle body with a motor drive unit to which the burr assembly of FIG. 1 can be coupled, with the handle body including an LCD screen for displaying operating parameters of device during use together with a joystick and mode control actuators on the handle.

FIG. 4 is an enlarged perspective view of the ceramic cutting member showing a manner of coupling the cutter to a distal end of the inner sleeve of the burr assembly.

FIG. 5A is a cross-sectional view of a cutting assembly similar to that of FIG. 2 taken along line 5A-5A showing the close tolerance between sharp cutting edges of a window in a ceramic cutting member and sharp lateral edges of the outer sleeve which provides a scissor-like cutting effect in soft tissue.

FIG. 5B is a cross-sectional view of the cutting assembly of FIG. 5A with the ceramic cutting member in a different rotational position than in FIG. 5A.

FIG. 6 is a perspective view of another ceramic cutting member carried at the distal end of an inner sleeve with a somewhat rounded distal nose and deeper flutes than the cutting member of FIGS. 2 and 4, and with aspiration openings or ports formed in the flutes.

FIG. 7 is a perspective view of another ceramic cutting member with cutting edges that extend around a distal nose of the cutter together with an aspiration window in the shaft portion and aspiration openings in the flutes.

FIG. 8 is a perspective view of a ceramic housing carried at the distal end of the outer sleeve.

FIG. 9 is a perspective of another variation of a ceramic member with cutting edges that includes an aspiration window and an electrode arrangement positioned distal to the window.

FIG. 10 is an elevational view of a ceramic member and shaft of FIG. 9 showing the width and position of the electrode arrangement in relation to the window.

FIG. 11 is an end view of a ceramic member of FIGS. 9-10 the outward periphery of the electrode arrangement in relation to the rotational periphery of the cutting edges of the ceramic member.

FIG. 12A is a schematic view of the working end and ceramic cutting member of FIGS. 9-11 illustrating a step in a method of use.

FIG. 12B is another view of the working end of FIG. 12A illustrating a subsequent step in a method of use to ablate a tissue surface.

FIG. 12C is a view of the working end of FIG. 12A illustrating a method of tissue resection and aspiration of tissue chips to rapidly remove volumes of tissue.

FIG. 13A is an elevational view of an alternative ceramic member and shaft similar to that of FIG. 9 illustrating an electrode variation.

FIG. 13B is an elevational view of another ceramic member similar to that of FIG. 12A illustrating another electrode variation.

FIG. 13C is an elevational view of another ceramic member similar to that of FIGS. 12A-12B illustrating another electrode variation.

FIG. 14 is a perspective view of an alternative working end and ceramic cutting member with an electrode partly encircling a distal portion of an aspiration window.

FIG. 15A is an elevational view of a working end variation with an electrode arrangement partly encircling a distal end of the aspiration window.

FIG. 15B is an elevational view of another working end variation with an electrode positioned adjacent a distal end of the aspiration window.

FIG. 16 is a perspective view of a variation of a working end and ceramic member with an electrode adjacent a distal end of an aspiration window having a sharp lateral edge for cutting tissue.

FIG. 17 is a perspective view of a variation of a working end and ceramic member with four cutting edges and an electrode adjacent a distal end of an aspiration window.

FIG. 18 is perspective view of an arthroscopic system including a control and power console, a footswitch and a re-usable motor carrying a motor drive unit.

FIG. 19 is an enlarged sectional view of the distal end of the handle of FIG. 18 showing first and second electrical contacts therein for coupling RF energy to a disposable RF probe.

FIG. 20 is a perspective view of a disposable RF probe of the type that couples to the re-useable handle of FIGS. 18-19.

FIG. 21 is a sectional perspective view of a proximal hub portion of the disposable RF probe of FIG. 20.

FIG. 22 is a sectional view of a variation of the hub of FIG. 21 which includes a fluid trap for collecting any conductive fluid migrating proximally in the hub.

FIG. 23 is a view of an arthroscopic handpiece and shaver blade showing a plurality of Hall sensors in the handpiece and magnets in the shaver blade that allow for multiple control functions, including shaver blade identification, up-down orientation of the shaver blade in the handpiece, tachometer functionality for determining rotational speed, and a rotational or reciprocation stop mechanism for stopping the moveable shaver blade component in a pre-selected position.

FIG. 24 is a perspective view of a metal collar further shown in FIG. 24 adapted for mechanically interlocking the ceramic cutting member with the metal sleeve.

FIG. 25 is a partly sectional view of the metal collar of FIG. 24 in its final position to form a mechanical interlock between the ceramic cutting member and the metal sleeve.

### DETAILED DESCRIPTION OF THE INVENTION

The present invention relates to bone cutting and removal devices and related methods of use. Several variations of the invention will now be described to provide an overall understanding of the principles of the form, function and methods of use of the devices disclosed herein. In general, the present disclosure provides for an arthroscopic cutter or burr assembly for cutting or abrading bone that is disposable and is configured for detachable coupling to a non-disposable handle and motor drive component. This description of the general principles of this invention is not meant to limit the inventive concepts in the appended claims.

In general, the present invention provides a high-speed rotating ceramic cutter or burr that is configured for use in many arthroscopic surgical applications, including but not limited to treating bone in shoulders, knees, hips, wrists, ankles and the spine. More in particular, the device includes a cutting member that is fabricated entirely of a ceramic material that is extremely hard and durable, as described in detail below. A motor drive is operatively coupled to the ceramic cutter to rotate the burr edges at speeds ranging from 3,000 rpm to 20,000 rpm.

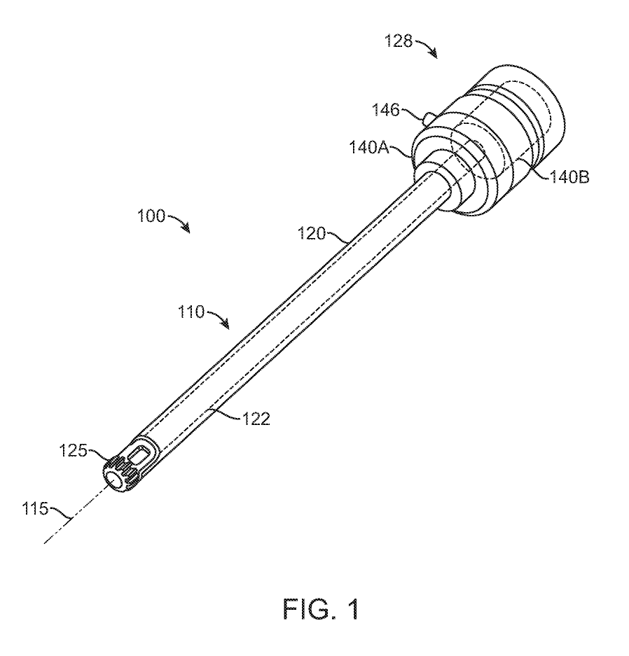

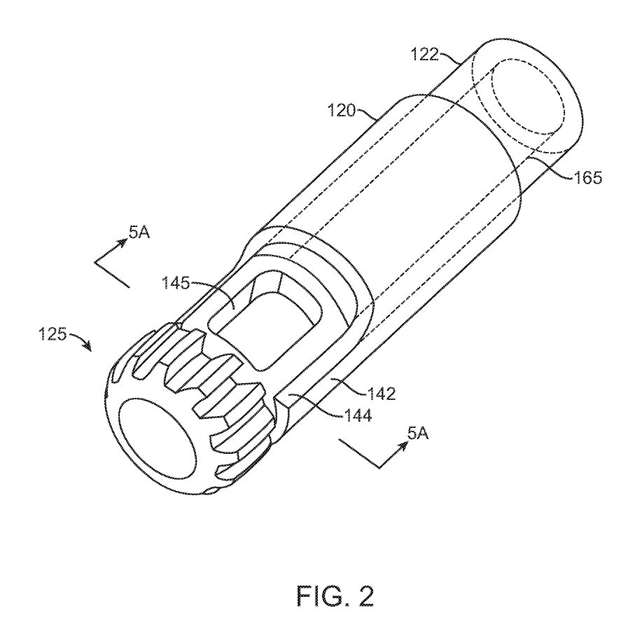

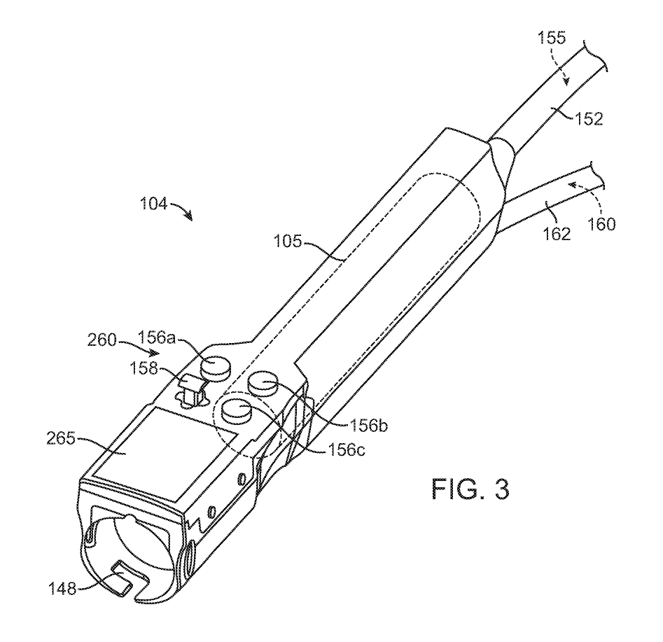

In one variation shown in FIGS. 1-2, an arthroscopic cutter or burr assembly 100 is provided for cutting and removing hard tissue, which operates in a manner similar to commercially available metals shavers and burrs. FIG. 1 shows disposable burr assembly 100 that is adapted for detachable coupling to a handle 104 and motor drive unit 105 therein as shown in FIG. 3.

The cutter assembly 100 has a shaft 110 extending along longitudinal axis 115 that comprises an outer sleeve 120 and an inner sleeve 122 rotatably disposed therein with the inner sleeve 122 carrying a distal ceramic cutting member 125. The shaft 110 extends from a proximal hub assembly 128 wherein the outer sleeve 120 is coupled in a fixed manner to an outer hub 140A which can be an injection molded plastic, for example, with the outer sleeve 120 insert molded therein. The inner sleeve 122 is coupled to an inner hub 140B (phantom view) that is configured for coupling to the motor drive unit 105 (FIG. 3). The outer and inner sleeves 120 ands 122 typically can be a thin wall stainless steel tube, but other materials can be used such as ceramics, metals, plastics or combinations thereof.

Referring to FIG. 2, the outer sleeve 120 extends to distal sleeve region 142 that has an open end and cut-out 144 that is adapted to expose a window 145 in the ceramic cutting member 125 during a portion of the inner sleeve's rotation. Referring to FIGS. 1 and 3, the proximal hub 128 of the burr assembly 100 is configured with a J-lock, snap-fit feature, screw thread or other suitable feature for detachably locking the hub assembly 128 into the handle 104. As can be seen in FIG. 1, the outer hub 140A includes a projecting key 146 that is adapted to mate with a receiving J-lock slot 148 in the handle 104 (see FIG. 3).

In FIG. 3, it can be seen that the handle 104 is operatively coupled by electrical cable 152 to a controller 155 which controls the motor drive unit 105. Actuator buttons 156a, 156b or 156c on the handle 104 can be used to select operating modes, such as various rotational modes for the ceramic cutting member. In one variation, a joystick 158 be moved forward and backward to adjust the rotational speed of the ceramic cutting member 125. The rotational speed of the cutter can continuously adjustable, or can be adjusted in increments up to 20,000 rpm. FIG. 3 further shows that negative pressure source 160 is coupled to aspiration tubing 162 which communicates with a flow channel in the handle 104 and lumen 165 in inner sleeve 122 which extends to window 145 in the ceramic cutting member 125 (FIG. 2).

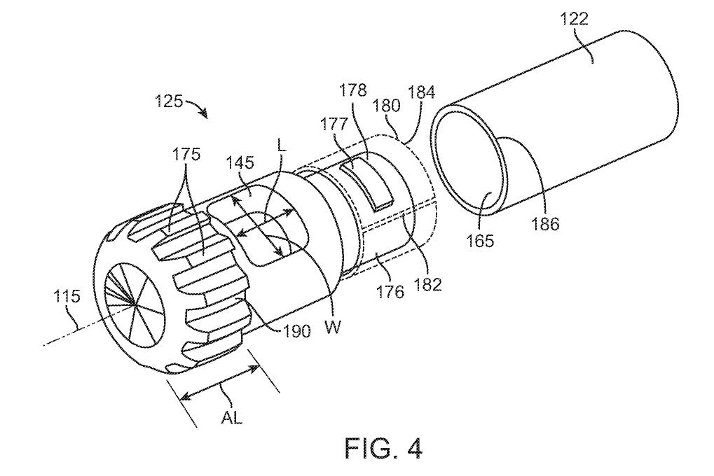

Now referring to FIGS. 2 and 4, the cutting member 125 comprises a ceramic body or monolith that is fabricated entirely of a technical ceramic material that has a very high hardness rating and a high fracture toughness rating, where “hardness” is measured on a Vickers scale and “fracture toughness” is measured in MPam^{1/2}. Fracture toughness refers to a property which describes the ability of a material containing a flaw or crack to resist further fracture and expresses a material's resistance to brittle fracture. The occurrence of flaws is not completely avoidable in the fabrication and processing of any components.

The authors evaluated technical ceramic materials and tested prototypes to determine which ceramics are best suited for the non-metal cutting member 125. When comparing the material hardness of the ceramic cutters of the invention to prior art metal cutters, it can easily be understood why typical stainless steel bone burrs are not optimal. Types 304 and 316 stainless steel have hardness ratings of 1.7 and 2.1, respectively, which is low and a fracture toughness ratings of 228 and 278, respectively, which is very high. Human bone has a hardness rating of 0.8, so a stainless steel cutter is only about 2.5 times harder than bone. The high fracture toughness of stainless steel provides ductile behavior which results in rapid cleaving and wear on sharp edges of a stainless steel cutting member. In contrast, technical ceramic materials have a hardness ranging from approximately 10 to 15, which is five to six times greater than stainless steel and which is 10 to 15 times harder than cortical bone. As a result, the sharp cutting edges of a ceramic remain sharp and will not become dull when cutting bone. The fracture toughness of suitable ceramics ranges from about 5 to 13 which is sufficient to prevent any fracturing or chipping of the ceramic cutting edges. The authors determined that a hardness-to-fracture toughness ratio (”hardness-toughness ratio”) is a useful term for characterizing ceramic materials that are suitable for the invention as can be understood form the Chart A below, which lists hardness and fracture toughness of cortical bone, a 304 stainless steel, and several technical ceramic materials.

CHART A

| | | | Ratio |
|---|---|---|---|
| | Hard- | Fracture | Hardness to |
| | ness | Toughness | Fracture |
| | (GPa) | (MPam^{1/2}) | Toughness |
| Cortical bone | 0.8 | 12 | .07:1 |
| Stainless steel 304 | 2.1 | 228 | .01:1 |
| Yttria-stabilized zirconia (YTZP) | | | |
| YTZP 2000 (Superior Technical | 12.5 | 10 | 1.25:1 |
| Ceramics) | | | |
| YTZP 4000 (Superior Technical | 12.5 | 10 | 1.25:1 |
| Ceramics) | | | |
| YTZP (CoorsTek) | 13.0 | 13 | 1.00:1 |
| Magnesia stabilized zirconia (MSZ) | | | |
| Dura-Z ® (Superior Technical | 12.0 | 11 | 1.09:1 |
| Ceramics) | | | |
| MSZ 200 (CoorsTek) | 11.7 | 12 | 0.98:1 |
| Zirconia toughened alumina (ZTA) | | | |
| YTA-14 (Superior Technical | 14.0 | 5 | 2.80:1 |
| Ceramics) | | | |
| ZTA (CoorsTek) | 14.8 | 6 | 2.47:1 |
| Ceria stabilized zirconia | | | |
| CSZ (Superior Technical | 11.7 | 12 | 0.98:1 |
| Ceramics) | | | |
| Silicon Nitride | | | |
| SiN (Superior Technical | 15.0 | 6 | 2.50:1 |
| Ceramics) | | | |

As can be seen in Chart A, the hardness-toughness ratio for the listed ceramic materials ranges from 98× to 250× greater than the hardness-toughness ratio for stainless steel 304. In one aspect of the invention, a ceramic cutter for cutting hard tissue is provided that has a hardness-toughness ratio of at least 0.5:1, 0.8:1 or 1:1.

In one variation, the ceramic cutting member 125 is a form of zirconia. Zirconia-based ceramics have been widely used in dentistry and such materials were derived from structural ceramics used in aerospace and military armor. Such ceramics were modified to meet the additional requirements of biocompatibility and are doped with stabilizers to achieve high strength and fracture toughness. The types of ceramics used in the current invention have been used in dental implants, and technical details of such zirconia-based ceramics can be found in Volpato, et al., “Application of Zirconia in Dentistry: Biological, Mechanical and Optical Considerations”, Chapter 17 in Advances in Ceramics—Electric and Magnetic Ceramics, Bioceramics, Ceramics and Environment (2011).

In one variation, the ceramic cutting member 125 is fabricated of an yttria-stabilized zirconia as is known in the field of technical ceramics, and can be provided by CoorsTek Inc., 16000 Table Mountain Pkwy., Golden, Colo. 80403 or Superior Technical Ceramics Corp., 600 Industrial Park Rd., St. Albans City, Vt. 05478. Other technical ceramics that may be used consist of magnesia-stabilized zirconia, ceria-stabilized zirconia, zirconia toughened alumina and silicon nitride. In general, in one aspect of the invention, the monolithic ceramic cutting member 125 has a hardness rating of at least 8 Gpa (kg/mm²). In another aspect of the invention, the ceramic cutting member 125 has a fracture toughness of at least 2 MPam^{1/2}.

The fabrication of such ceramics or monoblock components are known in the art of technical ceramics, but have not been used in the field of arthroscopic or endoscopic cutting or resecting devices. Ceramic part fabrication includes molding, sintering and then heating the molded part at high temperatures over precise time intervals to transform a compressed ceramic powder into a ceramic monoblock which can provide the hardness range and fracture toughness range as described above. In one variation, the molded ceramic member part can have additional strengthening through hot isostatic pressing of the part. Following the ceramic fabrication process, a subsequent grinding process optionally may be used to sharpen the cutting edges 175 of the burr (see FIGS. 2 and 4).

In FIG. 4, it can be seen that in one variation, the proximal shaft portion 176 of cutting member 125 includes projecting elements 177 which are engaged by receiving openings 178 in a stainless steel split collar 180 shown in phantom view. The split collar 180 can be attached around the shaft portion 176 and projecting elements 177 and then laser welded along weld line 182. Thereafter, proximal end 184 of collar 180 can be laser welded to the distal end 186 of stainless steel inner sleeve 122 to mechanically couple the ceramic body 125 to the metal inner sleeve 122. In another aspect of the invention, the ceramic material is selected to have a coefficient of thermal expansion between is less than 10 (1×10⁶/° C.) which can be close enough to the coefficient of thermal expansion of the metal sleeve 122 so that thermal stresses will be reduced in the mechanical coupling of the ceramic member 125 and sleeve 122 as just described. In another variation, a ceramic cutting member can be coupled to metal sleeve 122 by brazing, adhesives, threads or a combination thereof.

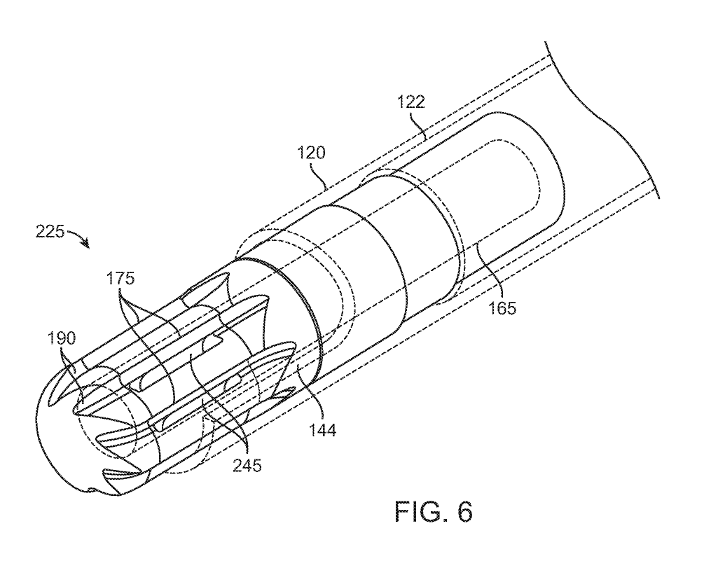

Referring to FIGS. 1 and 4, the ceramic cutting member 125 has window 145 therein which can extend over a radial angle of about 10° to 90° of the cutting member's shaft. In the variation of FIG. 1, the window is positioned proximally to the cutting edges 175, but in other variations, one or more windows or openings can be provided and such openings can extend in the flutes 190 (see FIG. 6) intermediate the cutting edges 175 or around a rounded distal nose of the ceramic cutting member 125. The length L of window 145 can range from 2 mm to 10 mm depending on the diameter and design of the ceramic member 125, with a width W of 1 mm to 10 mm.

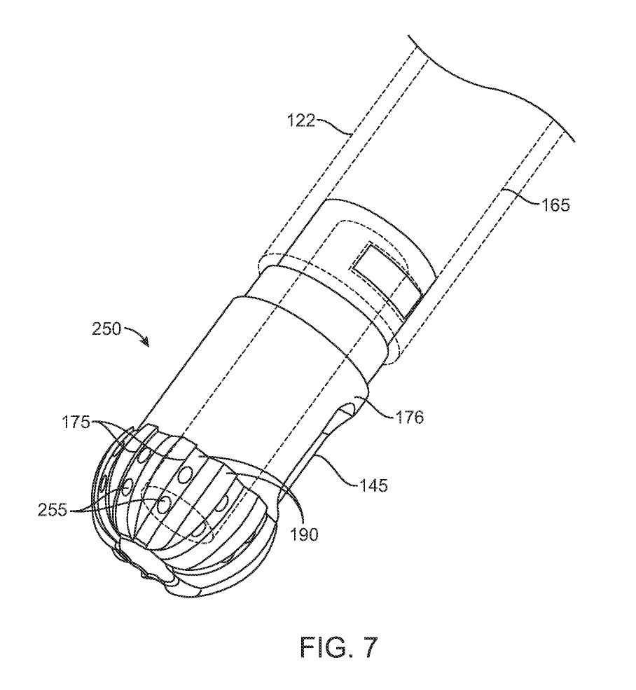

FIGS. 1 and 4 shows the ceramic burr or cutting member 125 with a plurality of sharp cutting edges 175 which can extend helically, axially, longitudinally or in a cross-hatched configuration around the cutting member, or any combination thereof. The number of cutting edges 175 ands intermediate flutes 190 can range from 2 to 100 with a flute depth ranging from 0.10 mm to 2.5 mm. In the variation shown in FIGS. 2 and 4, the outer surface or periphery of the cutting edges 175 is cylindrical, but such a surface or periphery can be angled relative to axis 115 or rounded as shown in FIGS. 6 and 7. The axial length AL of the cutting edges can range between 1 mm and 10 mm. While the cutting edges 175 as depicted in FIG. 4 are configured for optimal bone cutting or abrading in a single direction of rotation, it should be appreciated the that the controller 155 and motor drive 105 can be adapted to rotate the ceramic cutting member 125 in either rotational direction, or oscillate the cutting member back and forth in opposing rotational directions.

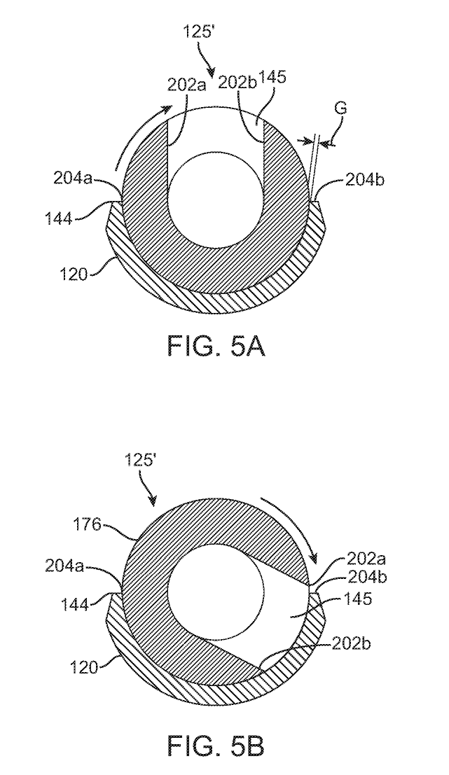

FIGS. 5A-5B illustrate a sectional view of the window 145 and shaft portion 176 of a ceramic cutting member 125′ that is very similar to the ceramic member 125 of FIGS. 2 and 4. In this variation, the ceramic cutting member has window 145 with one or both lateral sides configured with sharp cutting edges 202a and 202b which are adapted to resect tissue when rotated or oscillated within close proximity, or in scissor-like contact with, the lateral edges 204a and 204b of the sleeve walls in the cut-out portion 144 of the distal end of outer sleeve 120 (see FIG. 2). Thus, in general, the sharp edges of window 145 can function as a cutter or shaver for resecting soft tissue rather than hard tissue or bone. In this variation, there is effectively no open gap G between the sharp edges 202a and 202b of the ceramic cutting member 125′ and the sharp lateral edges 204a, 204b of the sleeve 120. In another variation, the gap G between the window cutting edges 202a, 202b and the sleeve edges 204a, 204b is less than about 0.020″, or less than 0.010″.

FIG. 6 illustrates another variation of ceramic cutting member 225 coupled to an inner sleeve 122 in phantom view. The ceramic cutting member again has a plurality of sharp cutting edges 175 and flutes 190 therebetween. The outer sleeve 120 and its distal opening and cut-out shape 144 are also shown in phantom view. In this variation, a plurality of windows or opening 245 are formed within the flutes 190 and communicate with the interior aspiration channel 165 in the ceramic member as described previously.

FIG. 7 illustrates another variation of ceramic cutting member 250 coupled to an inner sleeve 122 (phantom view) with the outer sleeve not shown. The ceramic cutting member 250 is very similar to the ceramic cutter 125 of FIGS. 1, 2 and 4, and again has a plurality of sharp cutting edges 175 and flutes 190 therebetween. In this variation, a plurality of windows or opening 255 are formed in the flutes 190 intermediate the cutting edges 175 and another window 145 is provided in a shaft portion 176 of ceramic member 225 as described previously. The openings 255 and window 145 communicate with the interior aspiration channel 165 in the ceramic member as described above.

It can be understood that the ceramic cutting members can eliminate the possibility of leaving metal particles in a treatment site. In one aspect of the invention, a method of preventing foreign particle induced inflammation in a bone treatment site comprises providing a rotatable cutter fabricated of a ceramic material having a hardness of at least 8 Gpa (kg/mm²) and/or a fracture toughness of at least 2 MPam^{1/2} and rotating the cutter to cut bone without leaving any foreign particles in the treatment site. The method includes removing the cut bone tissue from the treatment site through an aspiration channel in a cutting assembly.

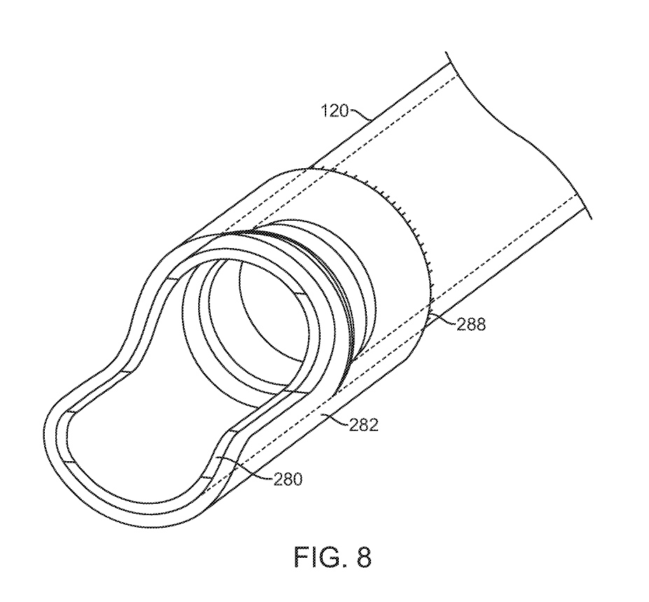

FIG. 8 illustrates variation of an outer sleeve assembly with the rotating ceramic cutter and inner sleeve not shown. In the previous variations, such as in FIGS. 1, 2 and 6, shaft portion 176 of the ceramic cutter 125 rotates in a metal outer sleeve 120. FIG. 8 illustrates another variation in which a ceramic cutter (not shown) would rotate in a ceramic housing 280. In this variation, the shaft or a ceramic cutter would thus rotate is a similar ceramic body which may be advantageous when operating a ceramic cutter at high rotational speeds. As can be seen in FIG. 8, a metal distal metal housing 282 is welded to the outer sleeve 120 along weld line 288. The distal metal housing 282 is shaped to support and provide strength to the inner ceramic housing 282.

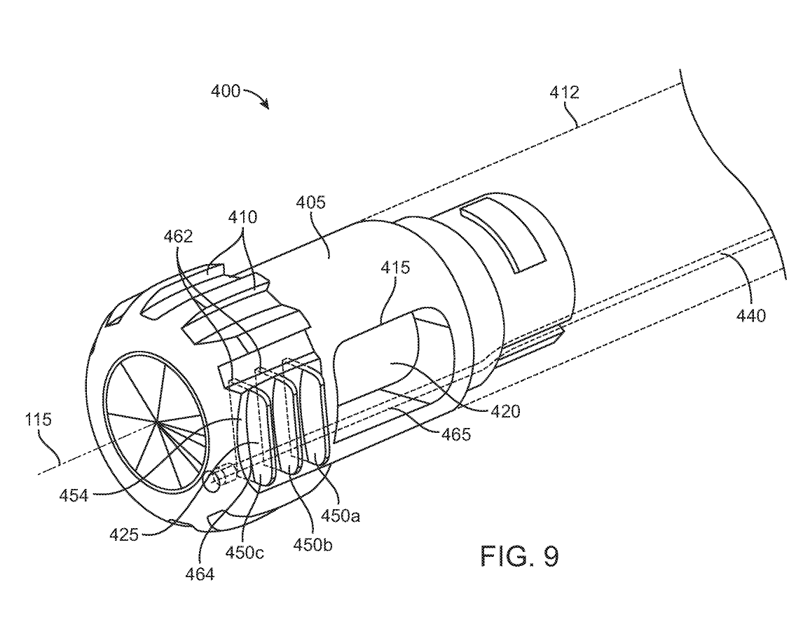

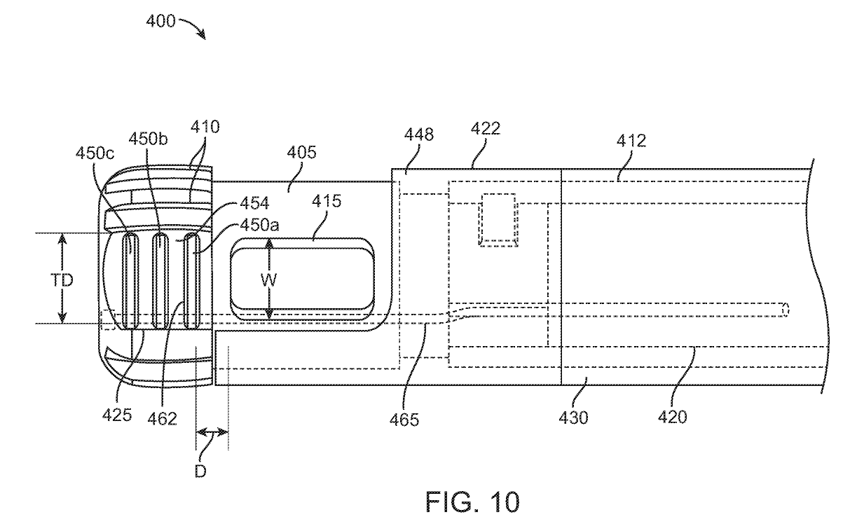

FIGS. 9-11 are views of an alternative tissue resecting assembly or working end 400 that includes a ceramic member 405 with cutting edges 410 in a form similar to that described previously. FIG. 9 illustrates the monolithic ceramic member 405 carried as a distal tip of a shaft or inner sleeve 412 as described in previous embodiments. The ceramic member 405 again has a window 415 that communicates with aspiration channel 420 in shaft 412 that is connected to negative pressure source 160 as described previously. The inner sleeve 412 is operatively coupled to a motor drive 105 and rotates in an outer sleeve 422 of the type shown in FIG. 2. The outer sleeve 422 is shown in FIG. 10.

In the variation illustrated in FIG. 9, the ceramic member 405 carries an electrode arrangement 425, or active electrode, having a single polarity that is operatively connected to an RF source 440. A return electrode, or second polarity electrode 430, is provided on the outer sleeve 422 as shown in FIG. 10. In one variation, the outer sleeve 422 can comprise an electrically conductive material such as stainless steel to thereby function as return electrode 445, with a distal portion of outer sleeve 422 is optionally covered by a thin insulative layer 448 such as parylene, to space apart the active electrode 425 from the return electrode 430.

The active electrode arrangement 425 can consist of a single conductive metal element or a plurality of metal elements as shown in FIGS. 9 and 10. In one variation shown in FIG. 9, the plurality of electrode elements 450a, 450b and 450c extend transverse to the longitudinal axis 115 of ceramic member 405 and inner sleeve 412 and are slightly spaced apart in the ceramic member. In one variation shown in FIGS. 9 and 10, the active electrode 425 is spaced distance D from the distal edge 452 of window 415 which is less than 5 mm and often less than 2 mm for reasons described below. The width W and length L of window 415 can be the same as described in a previous embodiment with reference to FIG. 4.

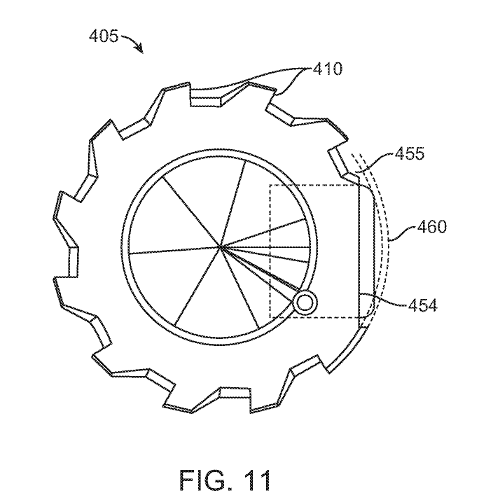

As can be seen in FIGS. 9 and 11, the electrode arrangement 425 is carried intermediate the cutting edges 410 of the ceramic member 405 in a flattened region 454 where the cutting edges 410 have been removed. As can be best understood from FIG. 11, the outer periphery 455 of active electrode 425 is within the cylindrical or rotational periphery of the cutting edges 410 when they rotate. In FIG. 11, the rotational periphery of the cutting edges is indicated at 460. The purpose of the electrode's outer periphery 455 being equal to, or inward from, the cutting edge periphery 460 during rotation is to allow the cutting edges 410 to rotate at high RPMs to engage and cut bone or other hard tissue without the surface or the electrode 425 contacting the targeted tissue.

FIG. 9 further illustrates a method of fabricating the ceramic member 405 with the electrode arrangement 425 carried therein. The molded ceramic member 405 is fabricated with slots 462 that receive the electrode elements 450a-450c, with the electrode elements fabricated from stainless steel, tungsten or a similar conductive material. Each electrode element 450a-450c has a bore 464 extending therethrough for receiving an elongated wire electrode element 465. As can be seen in FIG. 9, and the elongated wire electrode 465 can be inserted from the distal end of the ceramic member 405 through a channel in the ceramic member 405 and through the bores 464 in the electrode elements 450a-450c. The wire electrode 465 can extend through the shaft 412 and is coupled to the RF source 440. The wire electrode element 465 thus can be used as a means of mechanically locking the electrode elements 450a-450c in slots 462 and also as a means to deliver RF energy to the electrode 425.

Another aspect of the invention is illustrated in FIGS. 9-10 wherein it can be seen that the electrode arrangement 425 has a transverse dimension TD relative to axis 115 that is substantial in comparison to the window width W as depicted in FIG. 10. In one variation, the electrode's transverse dimension TD is at least 50% of the window width W, or the transverse dimension TD is at least 80% of the window width W. In the variation of FIGS. 9-10, the electrode transverse dimension TD is 100% or more of the window width W. It has been found that tissue debris and byproducts from RF ablation are better captured and extracted by a window 415 that is wide when compared to the width of the RF plasma ablation being performed.

In general, the tissue resecting system comprises an elongated shaft with a distal tip comprising a ceramic member, a window in the ceramic member connected to an interior channel in the shaft and an electrode arrangement in the ceramic member positioned distal to the window and having a width that is at 50% of the width of the window, at 80% of the width of the window or at 100% of the width of the window. Further, the system includes a negative pressure source 160 in communication with the interior channel 420.

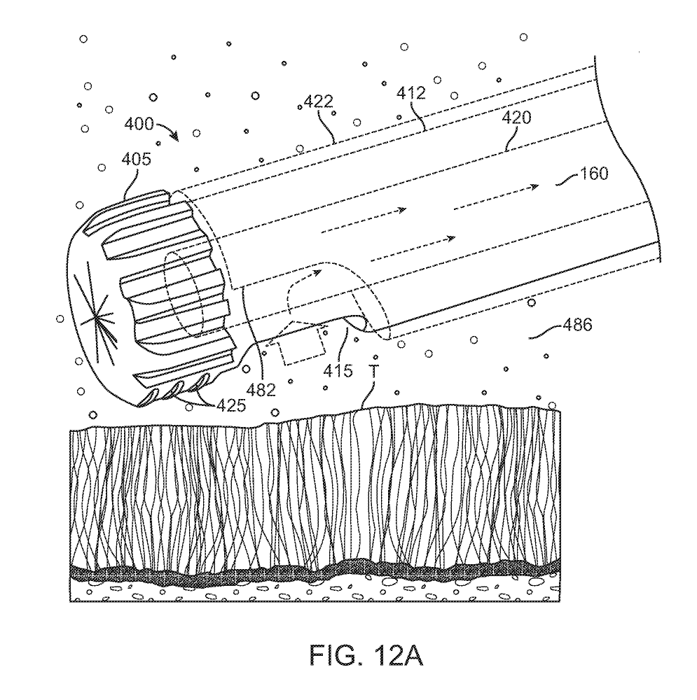

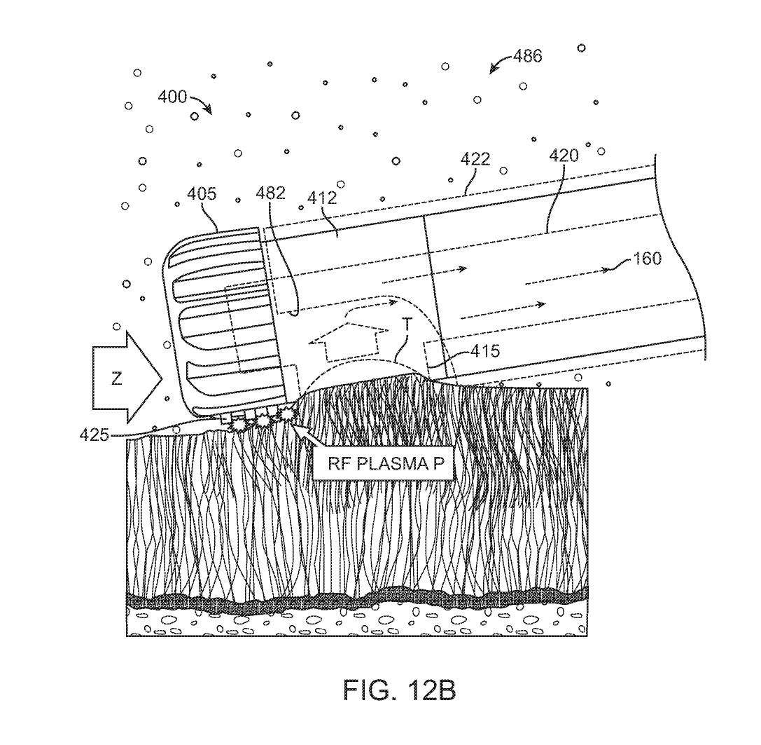

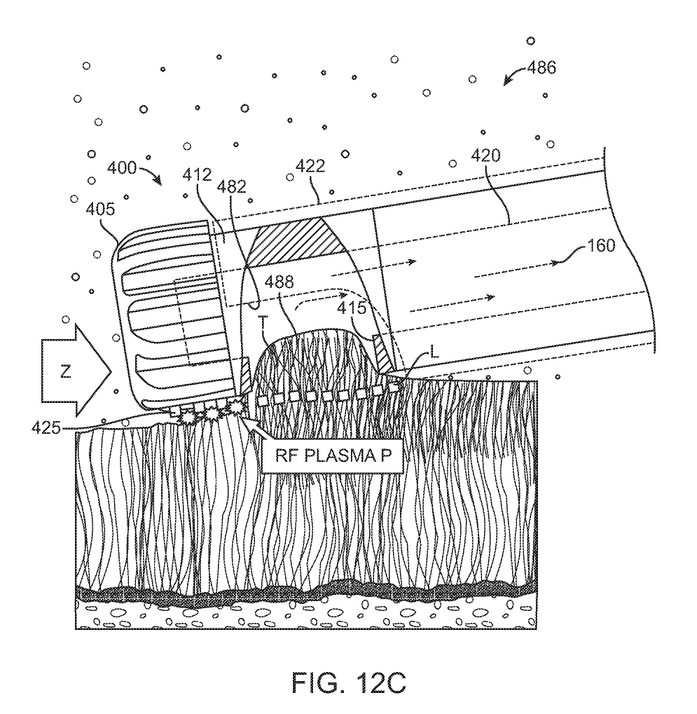

Now turning to FIGS. 12A-12C, a method of use of the resecting assembly 400 of FIG. 9 can be explained. In FIG. 12A, the system and a controller is operated to stop rotation of the ceramic member 405 in a selected position were the window 415 is exposed in the cut-out 482 of the open end of outer sleeve 422 shown in phantom view. In one variation, a controller algorithm can be adapted to stop the rotation of the ceramic 405 that uses a Hall sensor 484a in the handle 104 (see FIG. 3) that senses the rotation of a magnet 484b carried by inner sleeve hub 140B as shown in FIG. 2. The controller algorithm can receive signals from the Hall sensor which indicated the rotational position of the inner sleeve 412 and ceramic member relative to the outer sleeve 422. The magnet 484b can be positioned in the hub 140B (FIG. 2) so that when sensed by the Hall sensor, the controller algorithm can de-activate the motor drive 105 so as to stop the rotation of the inner sleeve in the selected position.

Under endoscopic vision, referring to FIG. 12B, the physician then can position the electrode arrangement 425 in contact with tissue targeted T for ablation and removal in a working space filled with fluid 486, such as a saline solution which enables RF plasma creation about the electrode. The negative pressure source 160 is activated prior to or contemporaneously with the step of delivering RF energy to electrode 425. Still referring to FIG. 12B, when the ceramic member 405 is positioned in contact with tissue and translated in the direction of arrow Z, the negative pressure source 160 suctions the targeted tissue into the window 415. At the same time, RF energy delivered to electrode arrangement 425 creates a plasma P as is known in the art to thereby ablate tissue. The ablation then will be very close to the window 415 so that tissue debris, fragments, detritus and byproducts will be aspirated along with fluid 486 through the window 415 and outwardly through the interior extraction channel 420 to a collection reservoir. In one method shown schematically in FIG. 12B, a light movement or translation of electrode arrangement 425 over the targeted tissue will ablate a surface layer of the tissue and aspirate away the tissue detritus.

FIG. 12C schematically illustrates a variation of a method which is of particular interest. It has been found if suitable downward pressure on the working end 400 is provided, then axial translation of working end 400 in the direction arrow Z in FIG. 12C, together with suitable negative pressure and the RF energy delivery will cause the plasma P to undercut the targeted tissue along line L that is suctioned into window 415 and then cut and scoop out a tissue chips indicated at 488. In effect, the working end 400 then can function more as a high volume tissue resecting device instead of, or in addition to, its ability to function as a surface ablation tool. In this method, the cutting or scooping of such tissue chips 488 would allow the chips to be entrained in outflows of fluid 486 and aspirated through the extraction channel 420. It has been found that this system with an outer shaft diameter of 7.5 mm, can perform a method of the invention can ablate, resect and remove tissue greater than 15 grams/min, greater than 20 grams/min, and greater than 25 grams/min.

In general, a method corresponding to the invention includes providing an elongated shaft with a working end 400 comprising an active electrode 425 carried adjacent to a window 415 that opens to an interior channel in the shaft which is connected to a negative pressure source, positioning the active electrode and window in contact with targeted tissue in a fluid-filled space, activating the negative pressure source to thereby suction targeted tissue into the window and delivering RF energy to the active electrode to ablate tissue while translating the working end across the targeted tissue. The method further comprises aspirating tissue debris through the interior channel 420. In a method, the working end 400 is translated to remove a surface portion of the targeted tissue. In a variation of the method, the working end 400 is translated to undercut the targeted tissue to thereby remove chips 488 of tissue.

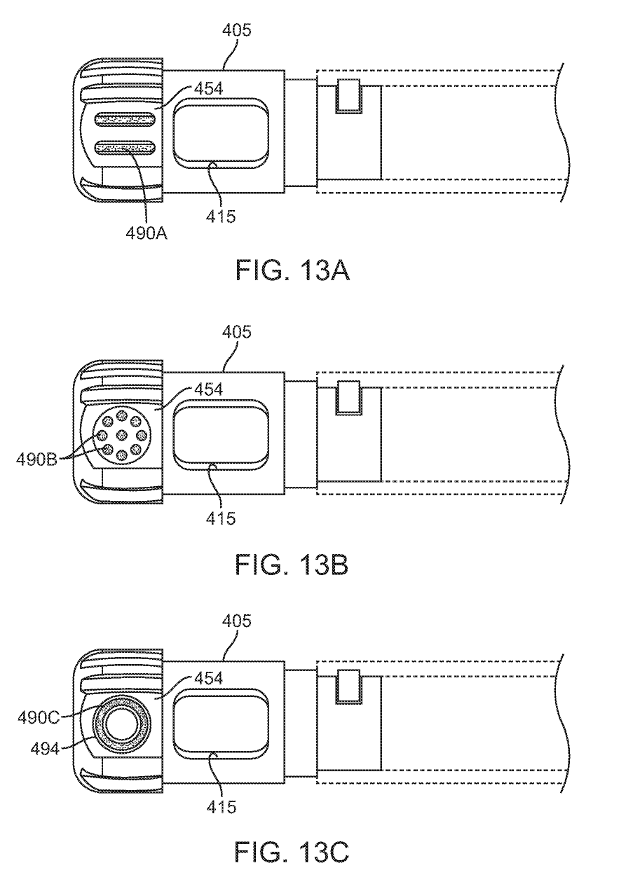

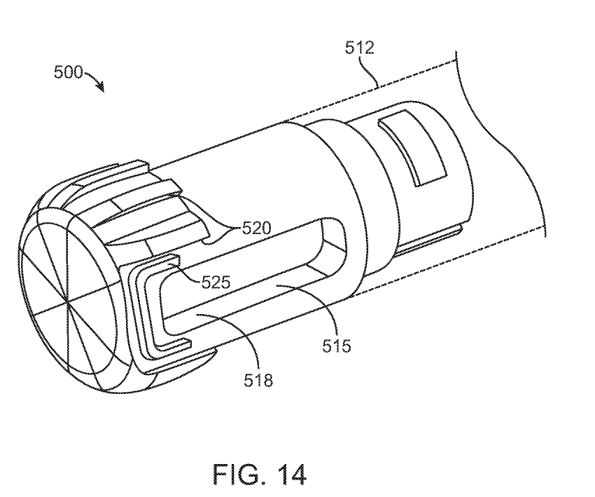

Now turning to FIGS. 13A-13C, other distal ceramic tips of cutting assemblies are illustrated that are similar to that of FIGS. 9-11, except the electrode configurations carried by the ceramic members 405 are varied. In FIG. 13A, the electrode 490A comprises one or more electrode elements extending generally axially distally from the window 415. FIG. 13B illustrates an electrode 490B that comprises a plurality of wire-like elements 492 projecting outwardly from surface 454. FIG. 13C shows electrode 490C that comprises a ring-like element that is partly recessed in a groove 494 in the ceramic body. All of these variations can produce an RF plasma that is effective for surface ablation of tissue, and are positioned adjacent to window 415 to allow aspiration of tissue detritus from the site.

FIG. 14 illustrates another variation of a distal ceramic tip 500 of an inner sleeve 512 that is similar to that of FIG. 9 except that the window 515 has a distal portion 518 that extends distally between the cutting edges 520, which is useful for aspirating tissue debris cut by high speed rotation of the cutting edges 520. Further, in the variation of FIG. 14, the electrode 525 encircles a distal portion 518 of window 515 which may be useful for removing tissue debris that is ablated by the electrode when the ceramic tip 500 is not rotated but translated over the targeted tissue as described above in relation to FIG. 12B. In another variation, a distal tip 500 as shown in FIG. 14 can be energized for RF ablation at the same time that the motor drive rotates back and forth (or oscillates) the ceramic member 500 in a radial arc ranging from 1° to 180° and more often from 10° to 90°.

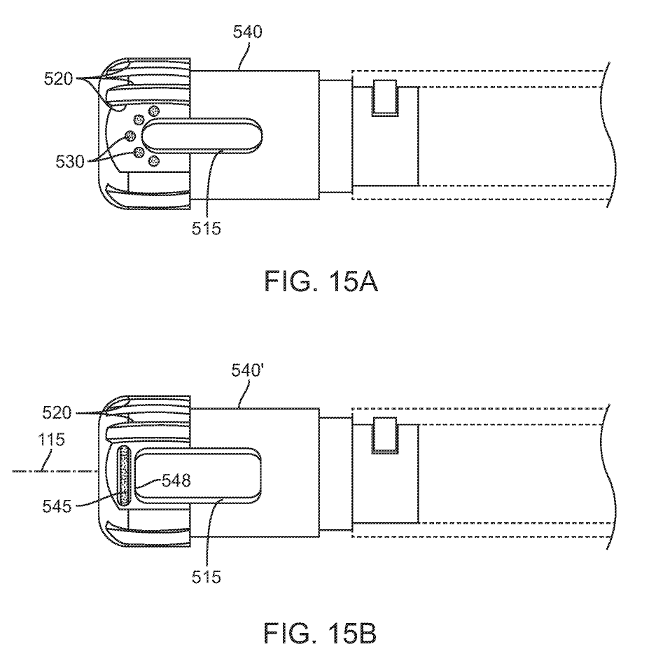

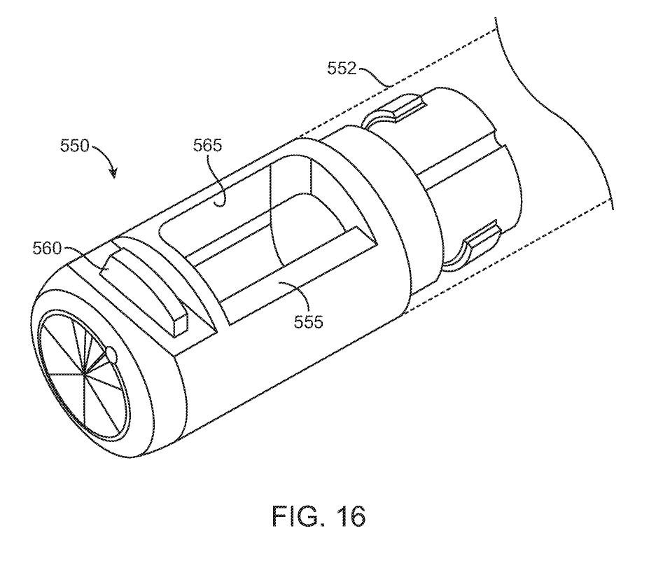

FIGS. 15A-15B illustrate other distal ceramic tips 540 and 540′ that are similar to that of FIG. 14 except the electrode configurations differ. In FIG. 15A, the window 515 has a distal portion 518 that again extends distally between the cutting edges 520, with electrode 530 comprising a plurality of projecting electrode elements that extend partly around the window 515. FIG. 15B shows a ceramic tip 540′ with window 515 having a distal portion 518 that again extends distally between the cutting edges 520. In this variation, the electrode 545 comprises a single blade element that extends transverse to axis 115 and is in close proximity to the distal end 548 of window 515.

FIG. 16 illustrates another variation of distal ceramic tip 550 of an inner sleeve 552 that is configured without the sharp cutting edges 410 of the embodiment of FIGS. 9-11. In other respects, the arrangement of the window 555 and the electrode 560 is the same as described previously. Further, the outer periphery of the electrode is similar to the outward surface of the ceramic tip 550. In the variation of FIG. 16, the window 555 has at least one sharp edge 565 for cutting soft tissue when the assembly is rotated at a suitable speed from 500 to 5,000 rpm. When the ceramic tip member 550 is maintained in a stationary position and translated over targeted tissue, the electrode 560 can be used to ablate surface layers of tissue as described above.

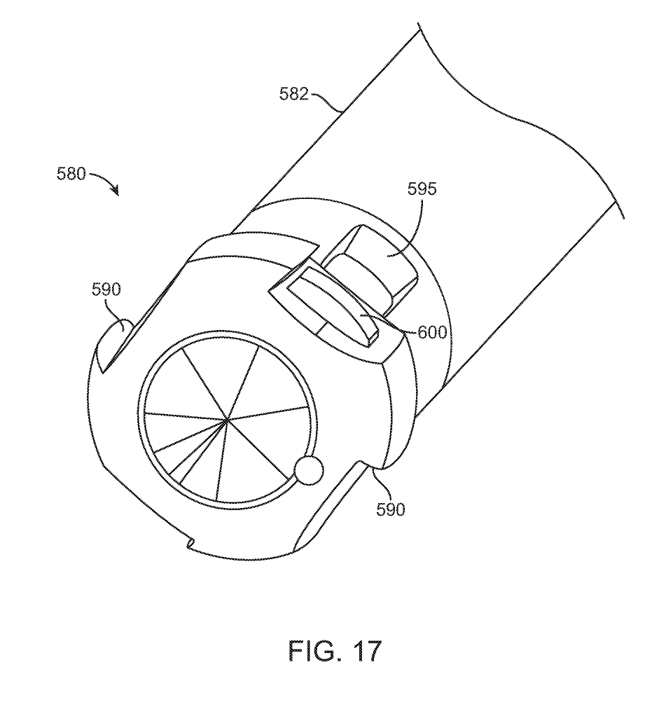

FIG. 17 depicts another variation of distal ceramic tip 580 coupled to an inner sleeve 582 that again has sharp burr edges or cutting edges 590 as in the embodiment of FIGS. 9-11. In this variation, the ceramic monolith has only 4 sharp edges 590 which has been found to work well for cutting bone at high RPMs, for example from 8,000 RPM to 20,000 RPM. In this variation, the arrangement of window 595 and electrode 600 is the same as described previously. Again, the outer periphery of electrode 595 is similar to the outward surface of the cutting edges 590.

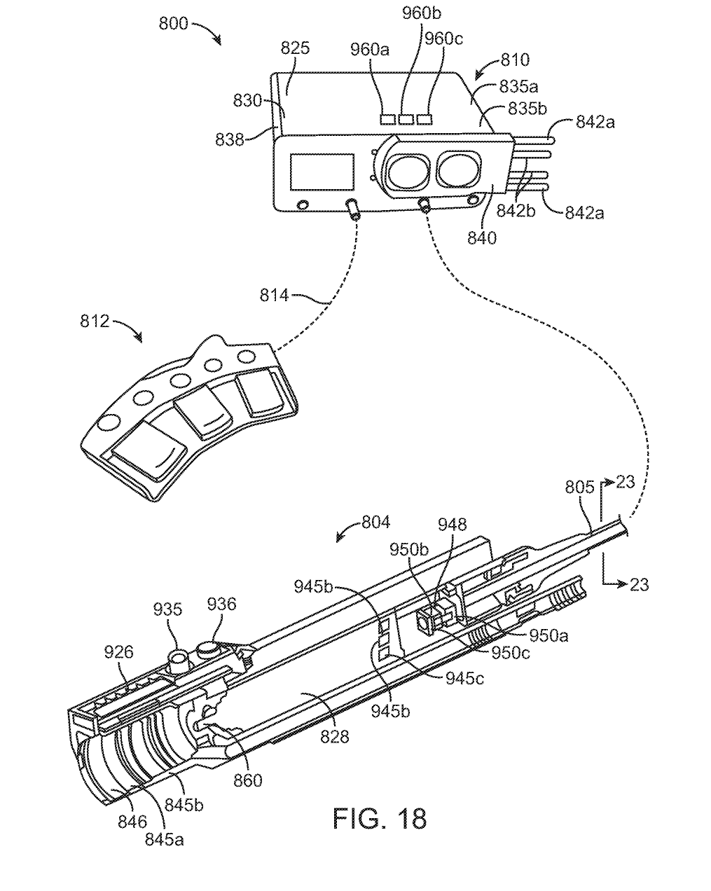

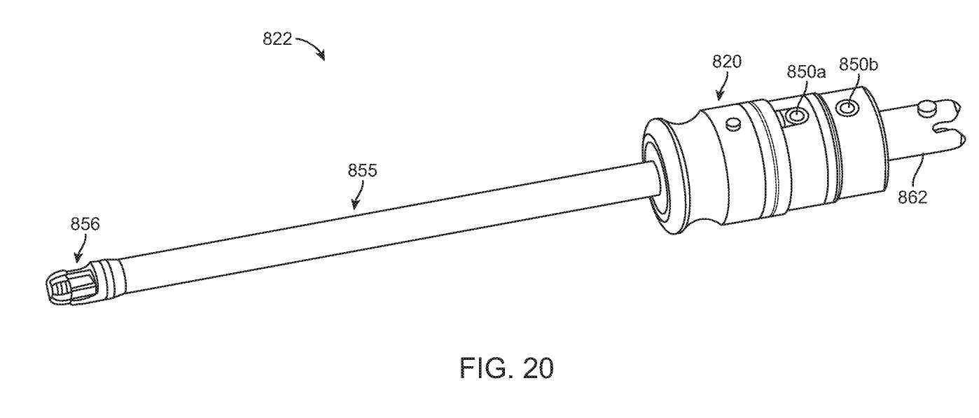

FIGS. 18-21 illustrate components of an arthroscopic system 800 including a re-usable handle 804 that is connected by a single umbilical cable or conduit 805 to a controller unit or console 810. Further, a footswitch 812 is connected by cable 814 to the console 810 for operating the system. As can be seen in FIGS. 18 and 20, the handle 804 is adapted to receive a proximal housing or hub 820 of a disposable shaver or probe 822 with RF functionality of the types shown in FIGS. 9-17 above.

In one variation, the console 810 of FIG. 18 includes an electrical power source 825 for operating the motor drive unit 828 in the handle 804, an RF source 830 for delivering RF energy to the RF electrodes of the disposable shaver 822, and dual peristaltic pumps 835A and 835B for operating the fluid management component of the system. The console 810 further carries a microprocessor or controller 838 with software to operate and integrate all the motor driven and RF functionality of the system. As can be seen in FIG. 18, a disposable cassette 840 carries inflow tubing 842a and outflow tubing 842b that cooperate with inflow and outflow peristaltic pumps in the console 810. The footswitch 812 in one variation includes switches for operating the motor drive unit 828, for operating the RF probe in a cutting mode with radiofrequency energy, and for operating the RF probe in a coagulation mode.

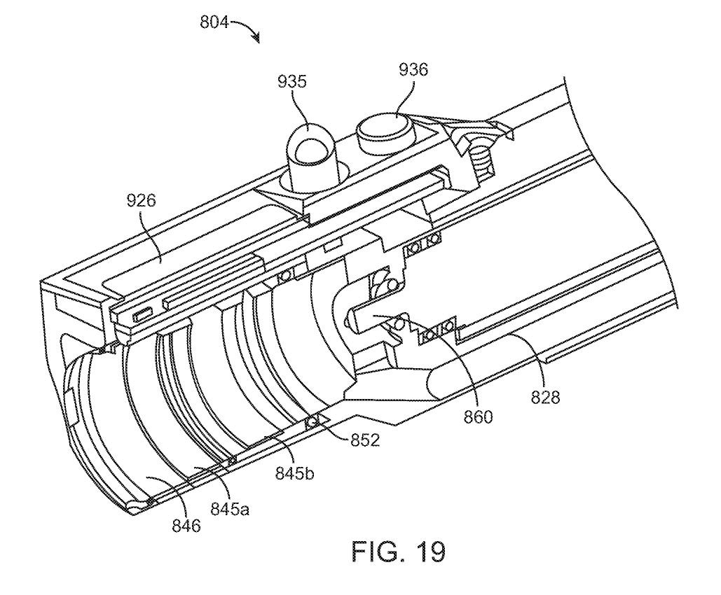

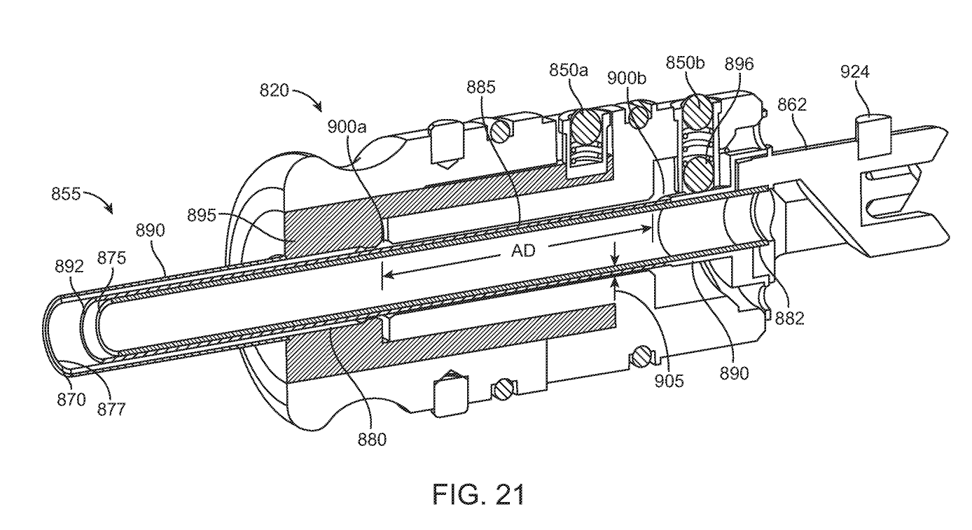

Of particular interest, the system of the invention includes a handle 804 with first and second electrical contacts 845A and 845B in a receiving passageway 846 of handle 804 (see FIG. 19) that cooperate with electrical contacts 850A and 850B in the proximal hub 820 of the disposable RF shaver 822 (see FIGS. 20-21). The RF shaver 822 has a shaft portion 855 that extends to working end 856 that carries a bi-polar electrode arrangement, of the type shown in FIGS. 9-17. This handle variation further includes providing all the necessary wiring and circuitry within the single conduit 805 that extends between handle 804 and the console 810. For example, the conduit 805 carries electrical leads for a 3-phase motor drive unit 828 in the handle 804, the electrical leads from the RF source 830 to the handle as well as a number of electrical leads for Hall sensors in the motor drive unit 828 that allow the controller 838 to control the operating parameters of the motor drive 828. In this variation, the handle 804 and the conduit 805 are a single component that can be easily sterilized, which is convenient for operating room personnel and economical for hospitals. As can be understood from FIG. 18, the conduit 805 is not detachable from the handle 804.

In the prior art, commercially available shavers that include an RF component utilize an independent RF electrical cable that couples directly to an exposed part of the prior art shaver hub that is exposed distally from the re-usable handle. In such prior art devices, the coupling of RF does not extend through the re-usable handle.

In order to provide a unitary handle 804 and conduit 805 for coupling to console 810 as shown in FIG. 18, a number of innovations are required for (i) coupling RF energy through the handle to the RF shaver, and (ii) in eliminating electrical interference among sensitive Hall sensor circuitry and the higher power current flows to the motor drive unit 828 and to the RF probe 822.

In one aspect of the invention, referring to FIG. 19, it can be seen that the electrical contacts 845A and 845B are cylindrical or partly cylindrical extending around the surface of the receiving passageway 846 of shaver hub 820 (see FIGS. 20-21). In use, it can be understood that such exposed electrical contacts 845A and 845B will be subject to alternating current corrosion, which is also known as stray current corrosion, which terms will be used interchangeably herein. Typically, stainless steel would be used for such electrical contacts. However, it has been found that stainless steel electrical contacts would have a very short lifetime in this application due to corrosion during use.

In this application, if stainless steel electrical contacts were used, alternating currents that would exit such stainless steel contact surfaces would be considered to consist of a blend of capacitive and resistive current. Such resistance is referred to as the polarization resistance, which is the transformation resistance that converts electron conductance into current conductance while capacitance makes up the electrochemical layer of the stainless steel surface. The capacitive portion of the current does not lead to corrosion, but causes reduction and oxidation of various chemical species on the metal surface. The resistive part of the current is the part that causes corrosion in the same manner as direct current corrosion. The association between the resistive and capacitive current components is known in alternating current corrosion and such resistance currents can leads to very rapid corrosion.

In one aspect of the invention, to prevent such alternating current corrosion, the electrical contacts 845A and 845B (FIG. 19) comprise materials that resist such corrosion. In one variation, the first and second electrical contacts 845A and 845B in handle 804 comprise a conductive material selected from the group of titanium, gold, silver, platinum, carbon, molybdenum, tungsten, zinc, Inconel, graphite, nickel or a combination thereof. The first and second electrical contacts 845A and 845B are spaced apart by at least 0.5 mm, 1.0 mm or 1.5 mm. Such electrical contacts can extend radially at least partly around the cylindrical passageway, or can extend in 360° around the cylindrical passageway 846.

In another variation, the hub 820 includes a fluid seal between the hub 820 and passageway 846, such as o-ring 852 in FIG. 19 carried by the handle 804. In another variation, one or more fluid seals can be carried by the hub 820, such as o-rings 854 and 856 shown in FIG. 21. As can be seen in FIG. 21, one such o-ring 856 can be positioned between the first and second contacts 845A and 845B in the hub 820 and 850A and 850B in the handle.

In general, the arthroscopic system corresponding to the invention provides a re-useable sterilizable shaver handle 804 with an integrated unitary power conduit 805 that carries electrical power for operating a motor drive unit 828 and a bi-polar RF probe 822, wherein the handle 804 includes first and second electrical contacts 845A and 845B that couple to corresponding electrical contacts 850A and 850B in a disposable RF probe 822.

In another aspect of the invention, the electrical contacts 845A and 845B in the handle are provided in a material that is resistant to alternating current corrosion.

In another aspect of the invention, the handle carries a motor drive unit with a rotating shaft 860 that engages a rotating coupler 862 in the hub 820, wherein the shaft 860 is plated or coated with a material resistant to alternating current corrosion.

Referring to FIGS. 20 and 21, another aspect of the invention relates to designs and mechanisms for effectively coupling RF energy from RF source 830 to working end 856 of the RF probe 822 through two thin-wall concentric, conductive sleeves that are assembled into the shaft 855 of the RF probe (see FIG. 21).

FIG. 21 is an enlarged sectional view of the hub 820 of RF probe 822 which illustrates the components and electrical pathways that enable RF delivery to the probe working end 856. More in particular, the shaft 855 comprises an outer sleeve 870 and a concentric inner sleeve 875 that is rotationally disposed in the bore 877 of the outer sleeve 870. Each of the outer sleeve 870 and inner sleeve 875 comprise a thin-wall conductive metal sleeve which carry RF current to and from spaced apart opposing polarity electrodes in the working end 856. In the variation shown in FIG. 21, the inner sleeve 875 comprises an electrical lead to the active electrode in a rotatable shaver component as shown, for example in FIG. 17. In FIG. 21, the outer sleeve 870 is stationary and fixed in hub 820 and has a distal end that comprises a return electrode as is known in the art.

As can be seen in FIG. 21, the outer and inner sleeves, 870 and 875, are separated by insulator layers as will be described below. The proximal end 880 of outer sleeve 870 is fixed in hub 820, for example over-molded with hub 820 of a nonconductive, plastic material. In FIG. 21, the proximal end 882 of the inner sleeve 875 is similarly fixed in a molded plastic coupler 862 that is adapted to mate with splines of shaft 860 of motor drive unit 828. Thus, it can be understood that the assembly of inner sleeve 875 and coupler 862 is adapted to rotate within a passageway 885 in the hub 820 and within bore 877 of outer sleeve 870.

The outer sleeve 870 has an exterior insulating layer 892, such as a heat shrink polymer, that extends distally from hub 820 over the shaft 855. The inner sleeve 875 similarly has a heat shrink polymer layer 892 over it outer surface which electrically separates the inner sleeve 875 from the outer sleeve 870 throughout the length of the shaft 855.

Now turning to the electrical pathways from the handle 804 to the outer and inner sleeves, 870 and 875, it can be seen that a first spring-loaded electrical contact 850A is provided in an exterior surface of hub 820 which is adapted to engage a corresponding electrical contact 845A in the handle 804 as shown in FIG. 19. The electrical contact 850A is connected to a conductive core component 895 within the hub 820 that in turn is coupled to the proximal end 880 of the outer sleeve 870.

FIG. 21 further shows a second spring-loaded electrical contact 850B in hub 820 that is adapted to deliver RF current to the rotating inner sleeve 875. In FIG. 21, the electrical contact 850B has a spring-loaded interior portion 896 that engages collar 890 which in turn is coupled to inner sleeve 875 and coupler 862.

Referring still to FIG. 21, can be seen that the hub assembly 820 and the outer sleeve 870 define a first proximal-most electrical region, herein called a first polarity region 900A, that is exposed to passageway 885 and obviously is electrically un-insulated from said passageway 885. Similarly, the assembly of inner sleeve 875 and collar 890 define a second polarity region 900B that is exposed to passageway 885 extending through hub 820.

It should be appreciated that the RF probe 822 is adapted for use with the working end 856 immersed a conductive saline solution. During use, it will be inevitable that saline will migrate, in part by capillary action, in the proximal direction passageway 885m that is in the annular space comprising the bore 877 of outer sleeve 870 and outward of inner sleeve 875 and its insulator layer 892. Although this annular space or passageway 885 is very small, saline solution still will migrate over the duration of an arthroscopic procedure, which can be from 5 minutes to an hour or more. As can be understood from FIG. 21, the saline eventually will migrate in passageway 885 in the hub 820 and thereafter form an electrically conductive path between the first and second opposing polarity regions 900A and 900B as shown in FIG. 21. If such a conductive saline path between such opposing polarity regions 900A and 900B is formed, it would comprise a short circuit and disrupt RF current flow to and from the working end 856. If such RF current flow through the short-circuit between regions 900A and 900B was insignificant, it could still cause unwanted heating in interior of hub 820. Thus, means are required to prevent or choke any potential RF current flow between the first and second opposing polarity regions 900A and 900B through passageway 885 in hub 820.

In one variation shown in FIG. 21, the longitudinal or axial dimension AD between the first and second opposing polarity regions 900A and 900B is selected to be large enough to substantially or entirely prevent electrical current flow between such regions 900A and 900B due to the high electrical resistance of such a potential current path. In a variation, the axial dimension is at least 0.5″, at least 0.6″, at least 0.8″ or at least 1.0″. In such a variation, it is also important to limit the radial dimension of the annular space or gap 905 between the inner and outer sleeves 870 and 875, which can further increases resistance to current flow between the first and second opposing polarity regions 900A and 900B. In a variation, the annular gap 905 has a radial dimension of less than 0.006″, less than 0.004″ or less than 0.002″.

By providing the selected axial dimension AD and radial dimension of the annular gap 905, the potential electrical pathway in a conductive fluid in passageway 885 and any potential unwanted current flow can be eliminated.

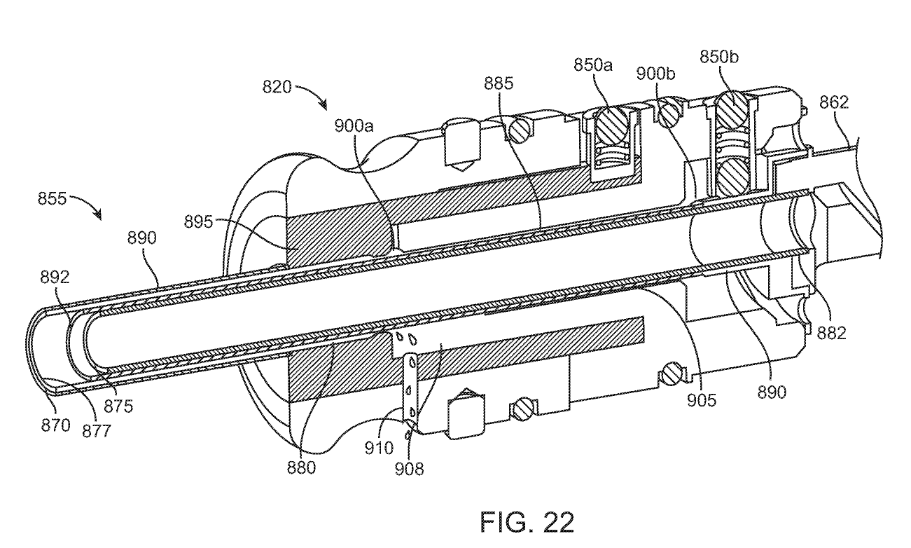

In other variations, other means can be provided to eliminate conductive saline solution from migrating in the annular gap 905. For example, FIG. 22 show a variation in which an enlarged annular or partly annular space or fluid trap 908 is provided to allow saline to drop by means of gravity into the space 908 and be collected therein. Such a space will prevent capillary action from assisting in the proximal migration of a conductive fluid in passageway 885. In a similar embodiment, still referring to FIG. 22, one or more apertures 910 can be provided in hub 820 to allow any saline in trap 808 to fall outwardly and be removed from the handle 804. In another variation, a desiccant material (not shown) can be exposed to the space 908 to absorb a conductive liquid and thus prevent an electrically conductive pathway between the first and second opposing polarity regions 900A and 900B (see FIG. 22).

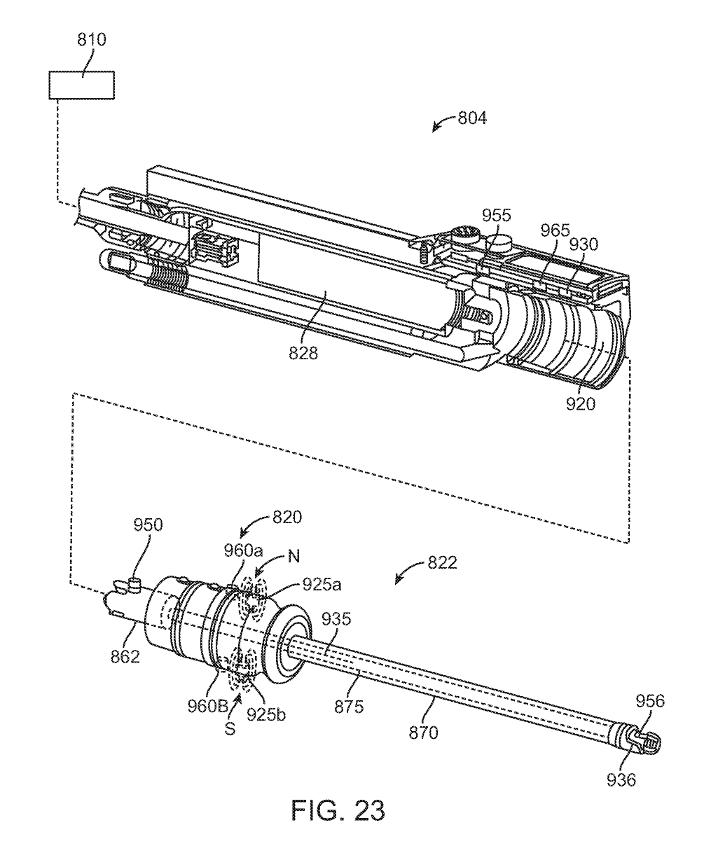

FIG. 23 illustrates arthroscopic handle or handpiece 804 and an exemplary shaver blade 822 (cf. FIG. 20) wherein the handpiece carries a plurality of Hall sensors and the shaver blade 822 carries a plurality of magnets that allow for multiple control functions in cooperation with controller algorithms, including (i) identification of the type of shaver blade received by the handpiece, (ii) the up-down orientation of the shaver blade in the handpiece which is needed to control the stop position of the rotating cutter relative to the handpiece in some types of shaver blades, (iii) tachometer functionality for determining the rotational speed of a rotating cutter in some embodiments, and (iv) a stop mechanism for stopping rotation of the moveable shaver blade component in a pre-selected final position.

In one system variation shown in FIG. 23, the shaver blade 822 of FIG. 20 is shown with a proximal hub 820 that is received by the receiver portion 920 of handpiece 804. The assembly of inner sleeve 875 and coupler 862 is adapted to rotate within the hub 820 and within bore of outer sleeve 870 as described previously.

In FIG. 23, a first and second magnet 925A and 925 B are carried in a surface portion of the hub 820. FIG. 23 further illustrates a Hall sensor 930 is carried by the handpiece 804 in axial alignment with the magnets 925A, 925B when the shaver blade 822 is coupled to handpiece 804. In one variation, the magnets can project outward from the hub 820 and function as J-lock elements that lock into cooperating grooves in receiver 920 of handpiece 804. In one aspect, the combination of the magnets 925A, 925B and Hall sensor 930 can be used to identify the type of shaver blade. For example, a product portfolio may have 5 types of shaver blades, and each such shaver blade can carry magnets 925A, 925B having a field strength specific to the blade type. Then, the Hall sensor 930 can be capable of providing a signal to the controller 810 indicating a range of the field strength, which can be compared to a library of field strengths each associated with a particular type of shaver blade. By this means, the controller can determine the shaver blade type and enable the software that controls the operating parameters of the motor 828, RF source and/or negative pressure source as required by the shaver blade type.

Still referring to FIG. 23, it can be seen that first and second magnets 925A and 925 B have different orientations of their North (N) and South (S) poles relative to central longitudinal axis 935 of the hub 820. In use, the physician may insert the shaver blade 822 into the handpiece receiver 920 with the outer sleeve cut-out opening 936 either “up” or “down”, with the J-locks locking the blade 822 in place in either the “up” or “down” orientation. By the terrms “up” and “down”, it is meant that the cut-out opening 936 of outer sleeve 870 is oriented either up or down relative to the handpiece 804. The physician needs the option of such up and down orientations for different procedures. In another aspect of the invention, in some types of shaver blades, the rotating inner sleeve 875 needs to stopped in a selected rotational position relative to the cut-out opening 936 in the outer sleeve 870. For this reason, the controller 810 needs to be able to determine the up-down orientation of the shaver blade 822 in the handpiece 804 as the stop mechanism may be linked to the relationship of another magnet 950 in coupler 862 and Hall sensor, which is further described below. In this aspect of the invention, the Hall sensor 930 then senses the pole of either magnet 925A and 925 B which is proximate the sensor and thereby can determine the up/down orientation of the shaver blade 822.

Referring again to FIG. 23, a third magnet 950 is carried by the rotating coupler 862. In FIG. 23, it can be seen that another Hall sensor 955 is carried by the handpiece 804 in axial alignment with the magnet 950 when the shaver blade 822 is coupled to the handpiece 804. It can be understood that each time the coupler 862 and magnet 925 rotates in 360°, the Hall sensor 955 will sense the magnet's field and can signal the controller 810 of each rotation and then a software tachometer algorithm can calculate and optionally display the RPM of the cutting member.

In another aspect of the invention, the magnet 950 and Hall sensor 955 are used in a set of controller algorithms to stop the rotation of the cutting member in a pre-selected rotational position, for example, with an inner sleeve window 956 exposed in the cut-out opening 936 of outer sleeve 870 (see FIG. 23).

As can be understood from FIG. 23, the controller 810 can always determine in real time the orientation of the window 956 (or other feature such as an electrode) of the inner sleeve 875 relative to the outer sleeve 870 by means of the Hall sensor 955 sensing the rotation of magnet 950. The controller algorithms can further calculate in real time the rotational angle of the window 956 away from the magnet/Hall sensor interface since the rotational speed is calculated by the algorithms.

In one variation, the stop mechanism of the invention uses (i) a dynamic braking method and algorithm used to stop the rotation of the inner sleeve in an initial position, and (2) in combination with a secondary “checking” algorithm that checks the initial stop position attained with the dynamic braking algorithm which is then followed by a slight reverse (or forward) rotation of the inner sleeve 875 as needed to position the inner sleeve with 0°-5° of the targeted stop point. Dynamic braking may typically stop the inner sleeve rotation with a variance of up to about 15° of the targeted stop point, but this can vary even further when different types of tissue are being cut and impeding rotation of the cutting member, and also depending on whether the physician has completely disengaged the cutting member from the tissue interface when the motor is de-activated. Therefore, dynamic braking alone cannot assure that the stop position is within the desired variance.

As background, the concept of dynamic braking is described in the following literature: https://www.ab.com/support/abdrives/documentation/techpapers/RegenOverview01.pdf and http://literature.rockwellautomation.com/idc/groups/literature/documents/wp/drives-wp004-en-p.pdf. Basically, a dynamic braking system provides a chopper transistor on the DC bus of the AC PWM drive that feeds a power resistor that transforms the regenerative electrical energy into heat energy. The heat energy is dissipated into the local environment. This process is generally called “dynamic braking” with the chopper transistor and related control and components called the “chopper module” and the power resistor called the “dynamic brake resistor”. The entire assembly of chopper module with dynamic brake resistor is sometimes referred to as the “dynamic brake module”. The dynamic brake resistor allows any magnetic energy stored in the parasitic inductance of that circuit to be safely dissipated during turn off of the chopper transistor.

The method is called dynamic braking because the amount of braking torque that can be applied is dynamically changing as the load decelerates. In other words, the braking energy is a function of the kinetic energy in the spinning mass and as it declines, so does the braking capacity. So the faster it is spinning or the more inertia it has, the harder you can apply the brakes to it, but as it slows, you run into the law of diminishing returns and at some point, you actually have no braking power left.

The braking method corresponding to the invention improves upon dynamic braking by adding an additional controller “checking” algorithm that calculates the position of magnet 950 relative to Hall sensor 955 after the inner sleeve 875 has stopped rotating which can be called an initial stop position. Thereafter, the algorithm instantly actuates the motor in reverse (or forward) to adjust the rotation direction of inner sleeve 875 to then stop rotation of the inner sleeve 875 exactly in the desired rotational position. It has been found that by using dynamic braking plus the checking algorithm, the inner sleeve 875 can be positioned within 0° to 5° of the targeted rotational orientation, or at the targeted orientation with 0° variance. In other words, in one variation, the window 956 of inner sleeve 875 can be precisely positioned within the cut-out opening 936 of the outer sleeve 870 (see FIG. 23).

Referring again to FIG. 23, additional magnets 960A and 960B are shown in phantom view in the shaver blade 822 and can cooperate with another Hall sensor 965 in handpiece 104 to allow for an additional signal for identification of shaver blades types. For example, with magnets 925A, 925B and Hall sensor 930 used for blade type identification, the various magnetic strengths may be stratified into 4 to 10 ranges that can be identified by Hall sensor 930 thus allowing for the identification of 4 to 10 blade types. By using additional magnets 960A, 960B wherein one of which would pass by Hall sensor 965 when the blade is inserted into receiver 920 of handpiece 104 (either up or down), a class of 4 to 10 blade types can be identified, and then the signal from either magnet 925A or 925B read by Hall sensor 930 can identify 4 to 10 sub-types allowing for a wider range of blade identification.

In general, an arthroscopic system comprises a handpiece carrying a motor, first and second types of shaver blades each having a proximal hub and a shaft extending about a longitudinal axis to a working end, each said hub adapted to be received by a receiver of the handpiece, at least one first magnet in the hub of said first shaver blade type having first magnetic parameters, at least one second magnet in the hub of said second shaver blade type having second magnetic parameters, and a sensor in the handpiece coupled to a controller configured to distinguish between the first and second magnetic parameters to identify the shaver blade type received by the handpiece receiver. In this embodiment, the sensor is a Hall sensor and the magnetic parameter can be magnetic field strength or an orientation of poles of the magnets.

In a variation, the arthroscopic system has a controller 810 configured to allow or disallow selected operating parameters and programs based on the shaver blade type that is identified. The operating parameters are at least one of rotation of a cutting surface, oscillation of a cutting surface, reciprocation of a cutting surface, speed of rotation, oscillation or reciprocation, RF energy delivery to a cutting surface, and up/down orientation of a shaver blade relative to a handpiece.

In another aspect of the invention, a disposable arthroscopic shaver blade comprises a proximal hub with a shaft extending about a longitudinal axis to a working end, a first magnet carried by the hub with the poles of said first magnet having a first orientation relative to the longitudinal axis, and a second magnet carried by the hub with the poles of said second magnet having a second relative to the longitudinal axis that differs from said first orientation. The first and second magnets are disposed on opposing sides of the hub.

In another aspect of the invention, an arthroscopic system comprises a handpiece carrying a motor, a shaver blade having a proximal hub and a shaft extending about a longitudinal axis to a working end, the hub adapted to be received by a receiver of the handpiece, first and second magnets having first and second respective magnetic parameters carried in the hub, and a sensor in the handpiece adapted to sense a selected range of magnetic parameters in proximity of said sensor.

In another aspect of the invention, an arthroscopic system comprises a handpiece carrying a motor, a shaver blade having a proximal hub and a shaft extending about a longitudinal axis to a working end, the hub adapted to be received by a receiver of the handpiece, a magnet carried in a rotating coupling carried by the hub, and a sensor in the handpiece adapted to sense the rotation of the magnet and coupling as a signal from which rotational speed can be calculated by a controller.

In another aspect of the invention, an arthroscopic system comprises a probe having a motor driven inner member that disposed in a passageway in an outer sleeve, a controller operatively configured to control the motor to stop movement of the inner member relative to the outer sleeve in a pre-selected stop position, a first dynamic braking algorithm adapted to control the motor to a stop movement of the inner member in an initial stop position, and a second check algorithm adapted to control the motor to move the inner member from the initial stop position to the pre-selected stop position.

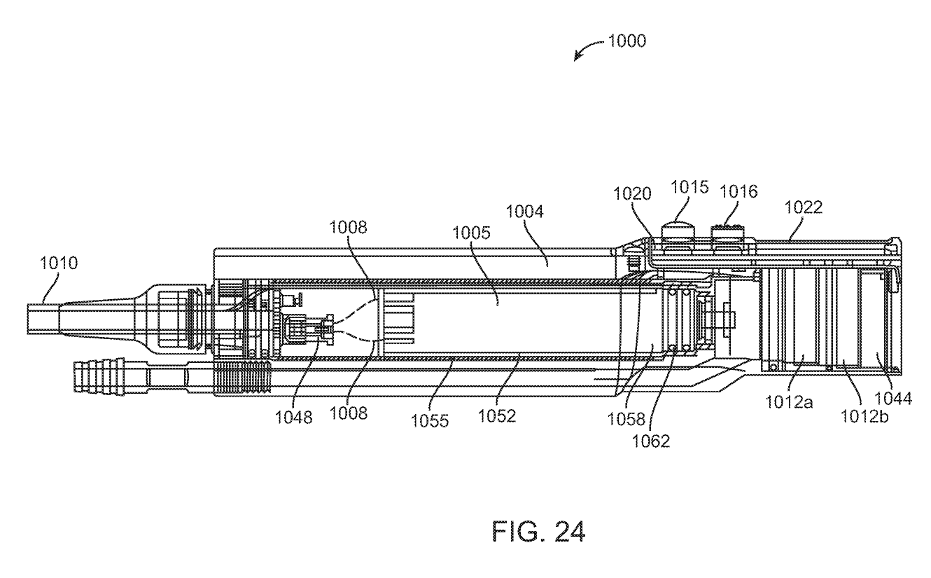

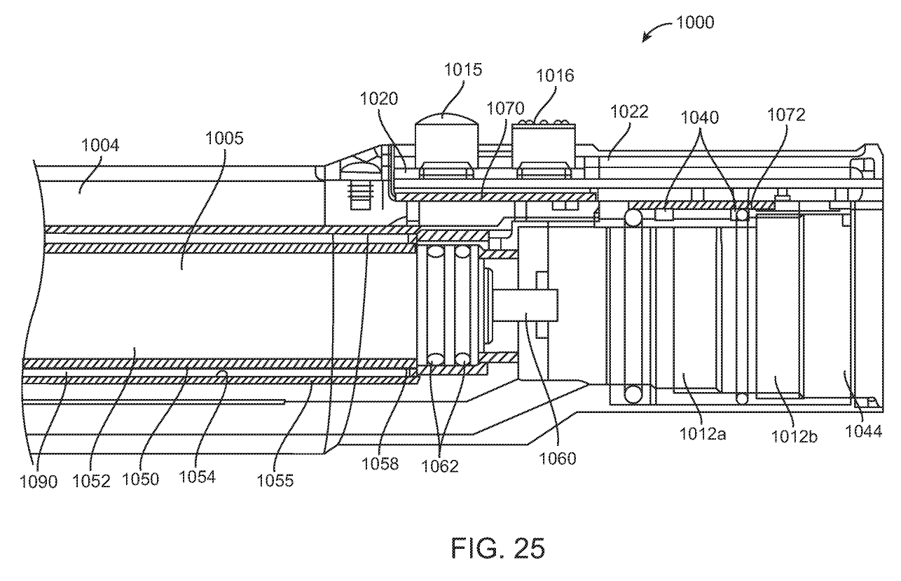

Now turning to FIGS. 24-25, another aspect of the invention is shown. A motorized handpiece 1000 is constructed with electrically insulating features configured to electrically isolate the handle to inhibit or prevent current leakage from interior electrical components to the handle body 1004. This is particularly important when the handle body is formed at least in part from an electrically conductive metal, such as aluminum, which has been selected based on its high thermal conductivity. As shown in FIGS. 24-25, the handle body 1004 typically is a heat conductive material such as aluminum or other heat conductive metal, to allow for effective heat dissipation from a motor 1005, which can generate a significant amount of heat during continuous operation for several minutes. In a variation, the handle body comprises a material having a thermal conductivity of greater than 50 W/(m·K), preferably greater than 100 W/(m·K), disposed in close proximity to the motor 1005. In addition, the motor can have a metal or other heat conductive shell which has an anodized or other electrically insulting layer formed at least partially thereover, typically being an aluminum shell having an anodized aluminum shell.

Referring to FIG. 24, as many heat conductive metals and other materials are also electrically conductive, any current leakage to the handle body 1004 from a defect in an electrical component could cause an unwanted electrical shock to the hand of the operator. The primary electrical component carried by the handle body 1004 is the motor 1005 which has electrical leads 1008 running through cable 1010. Other electrical components carried by the handle body 1004 include RF contacts 1012A and 1012B for delivering current to the disposable shaver, Hall sensors 1040 in a shaver-receiving passageway 1044 (FIG. 25), Schmitt triggers 1048, and a control panel 1020 which carries electrical on/off switches 1015, a joystick 1016, and an electronic display 1022.

To prevent electrical shocks, insulative layers and features are formed to electrically isolate the motor 1005 and other electrical components from an electrically conductive handle body 1004. FIG. 25 shows a first insulative layer 1050 comprising an anodized aluminum sleeve 1052 encasing the motor 1005. As is known in the art, a layer of anodization on sleeve 1052 can function as an electrical insulator. Further, the handle body 1004 typically carries a second insulative layer 1050 usually an anodized layer formed over a bore 1054 in an aluminum handle body. Two layers of electrically insulating anodized aluminum (1050, 1055) substantially encase the motor 1005 and provide redundant protection against electric shock.

Referring to FIG. 25, it can be seen that a motor shaft 1060 extends distally from a distal end 1058 of the motor 1005 and that a pair of O-ring seals 1062 at the distal end of the motor provide an additional insulative element which in combination with the first and second insulative layers 1050, 1055 can prevent electrical current leakage from the motor 1005 to the handle body 1004.

Referring again to FIG. 24, the anodized insulative layer 1055 in the bore 1054 that receives the motor 1005 also provides electrical insulation around the location of the Schmitt triggers 1048.

Referring to FIG. 25, another electrical insulator layer 1070, typically formed from a polymer or ceramic dielectric material, is positioned underneath the control panel 1020 which carries the on-off switches 1015, the joystick 1016 and electronic display 1022. FIG. 25 further illustrates an insulator layer 1072 which can be a ceramic or a polymer (e.g., Kapton) around the Hall sensors indicated at 1040. Further electrical insulation can be provided by an air gap 1090 disposed between an exterior surface of the motor 1005 (which is typically covered by the first insulating layer 1050) and an interior surface of the bore 1054 (FIG. 24) in in the aluminum handle body 1004.

A number of embodiments of the present invention have been described above in detail, and it should be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed is:

1. A motorized handpiece adapted for coupling to a disposable electrosurgical tool, said handpiece comprising:
a handle body having an exterior configured to be manually grasped and an interior, wherein the handle is formed at least in part from a material which is both thermally and electrically conductive;
electrical components including at least a motor and an RF contact within the interior of the handle body, said motor and said RF elected being configured for coupling to a motor drive and an RF electrode on the disposable electrosurgical tool; and
insulator elements disposed between the electrical components and surfaces of the interior of the handle body, wherein leakage current from the electrical components to the handle body is inhibited.

2. The motorized handpiece of claim 1, wherein the insulator elements are adapted to inhibit a potential leakage current above a threshold current of 5 mA, 10 mA, 25 mA, 50 mA, 100 mA or 200 mA.

3. The motorized handpiece of claim 1, further comprising an electrical cable extending from the handle body and configured to connect each electrical component to an external controller.

4. The motorized handpiece of claim 1, wherein the RF contact comprises first and second opposing polarity RF contacts adapted for coupling to bi-polar contacts on the disposable electrosurgical tool.

5. The motorized handpiece of claim 1, wherein the electrical components further include an electrical switch.

6. The motorized handpiece of claim 5, wherein the electrical components further include a plurality of electrical switches.

7. The motorized handpiece of claim 1, wherein the electrical components further include a control panel.

8. The motorized handpiece of claim 1, wherein the electrical components further include at least one Hall sensor.

9. The motorized handpiece of claim 1, wherein the electrical components further include at least one Schmitt trigger.

10. The motorized handpiece of claim 1, wherein one or more insulator element comprises an anodized layer of a metal.

11. The motorized handpiece of claim 1, wherein the handle body is formed at least is part from aluminum and one or more insulator elements comprises a layer of anodized aluminum formed over a surface of the interior.

12. The motorized handpiece of claim 1, wherein motor has an aluminum shell formed at least is part from aluminum and one or more insulator elements comprises a layer of anodized aluminum formed over a surface of the shell.

13. The motorized handpiece of claim 1, wherein one or more insulator elements comprises an air gap between an exterior of the motor and a surface of the interior of the handle body.

14. The motorized handpiece of claim 1, wherein one or more insulator elements comprises an O-ring.

15. The motorized handpiece of claim 7, wherein one insulator element comprises a polymer or ceramic dielectric material positioned underneath the control panel.

16. The motorized handpiece of claim 8, wherein one insulator element comprises a polymeric or ceramic layer between the Hall sensors and the handle body.

17. An arthroscopy handpiece, comprising:
a handle body;
a motor carried in an interior of the handle body, wherein the handle body comprises an electrically conductive material having a thermal conductivity of at least 50 W/(m·K) to carry heat away from the motor; and
electrical insulation between the handle body and the motor configured to inhibit a potential leakage current from the motor to the electrically conductive handle body.

18. The arthroscopy handpiece of claims 17, wherein the electrically conductive material having a thermal conductivity of at least 100 W/(m·K) to carry heat away from the motor.

19. The arthroscopy handpiece of claims 17, wherein the electrical insulation is adapted to inhibit a potential leakage current above a threshold current of 5 mA, 10 mA, 25 mA, 50 mA, 100 mA or 200 mA from the motor to the handle body.

20. The arthroscopy handpiece of claims 17, further comprising at least one electrical lead connected to the handle body.

21. The arthroscopy handpiece of claim 20, further comprising bi-polar RF contacts carried by the handle body with electrical leads extending from each contact away from the handle body, the RF contacts adapted for coupling to a disposable electrosurgical tool.

22. The arthroscopy handpiece of claims 17, wherein the handle body is formed at least is part from aluminum and the electrical insulation comprises a layer of anodized aluminum formed over a surface of the interior.

23. The arthroscopy handpiece of claims 17, wherein the motor has an aluminum shell formed at least is part from aluminum and the electrical insulation comprises a layer of anodized aluminum formed over a surface of the shell.

24. The arthroscopy handpiece of claims 17, wherein the electrical insulation comprises an air gap between an exterior of the motor and a surface of the interior of the handle body.

25. A motorized arthroscopy handpiece, comprising:
a handle body;
an electronic control panel carried by the handle body; and
at least one insulator disposed between the display and the handle body adapted to prevent a leakage current from the display to the handle body having a preselected threshold value.

26. The motorized handpiece of claim 25, wherein the preselected threshold value is 5 mA, 10 mA, 25 mA, 50 mA, 100 mA or 200 mA.

27. The motorized handpiece of claim 25, wherein the electronic control panel comprise an electronic display.

28. The motorized handpiece of claim 27, wherein the electronic display includes LCDs, LEDs and/or OLEDs.

29. The motorized handpiece of claim 25, wherein the at least one insulator comprises a polymeric or ceramic layer between the control panel and the handle body.
